SEQUENCE LISTING (55 sequences)

SEQ ID NO: 1 - DNA, 309 bp, Human immunodeficiency virus
  CDS (1)..(309)
atggagccagtagatcctcgtctagagccctggaagcatccaggaagtcagcctaaaactgcttgtaccaattgctattgtaaaaagtgttgctttcattgccaagtttgtttcataacaaaagccttaggcatctcctacggcaggaagaagcggagacagcgtcgaagacctcctcaaggcagtcagactcatcaagtttctctatcaaagcaacccacctcccaatcccgaggggacccgacaggcccgaaggaacagaagaagaaggtggagagagagacagagacagatccggtccatcagtga

SEQ ID NO: 2 - protein, 102 aa, Human immunodeficiency virus
MEPVDPRLEPWKHPGSQPKTACTNCYCKKCCFHCQVCFITKALGISYGRKKRRQRRRPPQGSQTHQVSLSKQPTSQSRGDPTGPKEQKKKVERETETDPVHQ

SEQ ID NO: 3 - DNA, 261 bp, Human immunodeficiency virus
  CDS (1)..(261)
atggagccagtagatcctcgtctagagccctggaagcatccaggaagtcagcctaaaactgcttgtaccaattgctattgtaaaaagtgttgctttcattgccaagtttgtttcataacaaaagccttaggcatctcctacggcaggaagaagcggagacagcgtcgaagacctcctcaaggcagtcagactcatcaagtttctctatcaaagcaacccacctcccaatcccgaggggacccgacaggcccgaaggaatag

SEQ ID NO: 4 - protein, 86 aa, Human immunodeficiency virus
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 5 - DNA, 261 bp, Human immunodeficiency virus
  CDS (1)..(261)
atggagccagtagatcctagactagagccctggaagcatccaggaagtcagcctaaaactgctggtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcagcccacctcccaatcccgaggggacccgacaggcccgaaggaatag

SEQ ID NO: 6 - protein, 86 aa, Human immunodeficiency virus
MEPVDPRLEPWKHPGSQPKTAGTNCYCKKCCFHCQVCFITKALGISYGRKKRRQRRRPPQGSQTHQVSLSKQPTSQSRGDPTGPKE

SEQ ID NO: 7 - DNA, 261 bp, Human immunodeficiency virus
  CDS (1)..(261)
atggagccagtagatcctagactagagccctggaagcatccaggaagtcagcctaaaactgcttgtaccaattgctattgtaaaaagtgttgctttcattgccaagtttgtttcataacagctgccttaggcatctcctatggcaggaagaagcggagacagcgacgaagacctcctcaaggcagtcagactcatcaagtttctctatcaaagcagcccacctcccaatcccgaggggacccgacaggcccgaaggaatag

SEQ ID NO: 8 - protein, 86 aa, Human immunodeficiency virus
MEPVDPRLEPWKHPGSQPKTACTNCYCKKCCFHCQVCFITAALGISYGRKKRRQRRRPPQGSQTHQVSLSKQPTSQSRGDPTGPKE

SEQ ID NO: 9 - DNA, 252 bp, Human immunodeficiency virus
  CDS (1)..(252)
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcagcccacctcccaatccccgacaggcccgaaggaatag

SEQ ID NO: 10 - protein, 83 aa, Human immunodeficiency virus
MEPVDPRLEPWKHPGSQPKTACTNCYCKKCCFHCQVCFITKALGISYGRKKRRQRRRPPQGSQTHQVSLSKQPTSQSPTGPKE

SEQ ID NO: 11 - DNA, 252 bp, Human immunodeficiency virus
  CDS (1)..(252)
atggagccagtagatcctagactagagccctggaagcatccaggaagtcagcctaaaactgcttgtaccaattgctattgtaaaaagtgttgctttcattgccaagtttgtttcataacagctgccttaggcatctcctatggcaggaagaagcggagacagcgacgaagacctcctcaaggcagtcagactcatcaagtttctctatcaaagcagcccacctcccaatccccgacaggcccgaaggaatag

SEQ ID NO: 12 - protein, 83 aa, Human immunodeficiency virus
MEPVDPRLEPWKHPGSQPKTACTNCYCKKCCFHCQVCFITAALGISYGRKKRRQRRRPPQGSQTHQVSLSKQPTSQSPTGPKE

SEQ ID NO: 13 - DNA, 306 bp, Human immunodeficiency virus
  CDS (1)..(306)
atggatccagtagatcctaacctagagccctggaaccatccgggaagtcagcctacaactgcttgtaacaagtgttactgtaaaaagtgttgctatcattgccaagtttgctttctgaacaaaggcttaggcatctcctatggcaggaagaagcggagacagcgacgaggaactcctcagagcagtaaggatcatcaaaatcctataccaaagcaacccataccccaaacccaaggggtctcgacaggcccggaagaatcgaagaagaaggtggagagcaaggcagagacagatcgattcgattag

SEQ ID NO: 14 - protein, 101 aa, Human immunodeficiency virus
MDPVDPNLEPWNHPGSQPTTACNKCYCKKCCYHCQVCFLNKGLGISYGRKKRRQRRGTPQSSKDHQNPIPKQPIPQTQGVSTGPEESKKKVESKAETDRFD

SEQ ID NO: 15 - DNA, 306 bp, Human immunodeficiency virus
  CDS (1)..(306)
atggagccagtagatcctagactagagccctggaagcatccaggaagtcagcctaagactgcttgtaccaattgctattgtaaaaagtgttgctttcattgccaagtttgtttcataacaaaaggcttaggcatctcctatggcaggaagaagcggagacagcgacgaagagctcctcaagacagtcagactcatcaagtttctctatcaaagcaacccgcctcccagccccgaggggacccgacaggcccgaaggaatcgaagaagaaggtggagagagagacagagacagatccggtcgattag

SEQ ID NO: 16 - protein, 101 aa, Human immunodeficiency virus
MEPVDPRLEPWKHPGSQPKTACTNCYCKKCCFHCQVCFITKGLGISYGRKKRRQRRRAPQDSQTHQVSLSKQPASQPRGDPTGPKESKKKVERETETDPVD

SEQ ID NO: 17 - DNA, 306 bp, Human immunodeficiency virus
  CDS (1)..(306)
atggagccagtagatcctaacctagagccctggaaccatccaggaagtcagcctaaaactgcttgtaataagtgttattgtaaacactgtagctatcattgtctagtttgctttcagacaaaaggcttaggcatttcctatggcaggaagaagcggagacagcgacgaagcgctcctccaagcagtgaggatcatcaaaatcttatatcaaagcaacccttaccccaaacccaaggggacccgacaggctcggaagaatcgaagaagaaggtggagagcaagacagagacagatccattcgattag

SEQ ID NO: 18 - protein, 101 aa, Human immunodeficiency virus
MEPVDPNLEPWNHPGSQPKTACNKCYCKHCSYHCLVCFQTKGLGISYGRKKRRQRRSAPPSSEDHQNLISKQPLPQTQGDPTGSEESKKKVESKTETDPFD

SEQ ID NO: 19 - DNA, 261 bp, Human immunodeficiency virus
  CDS (1)..(261)
atggatccagtagatcctaacctagagccctggaaccatccaggaagtcagcctaggactccttgtaacaagtgttattgtaaaaagtgttgctatcattgccaagtttgcttcataacgaaaggcttaggcatctcctatggcaggaagaagcggagacagcgacgaagacctcctcaaggcggtcaggctcatcaagatcctataccaaagcaaccctcctcccagccccgaggggacccgacaggcccgaaggaatag

SEQ ID NO: 20 - protein, 86 aa, Human immunodeficiency virus
MDPVDPNLEPWNHPGSQPRTPCNKCYCKKCCYHCQVCFITKGLGISYGRKKRRQRRRPPQGGQAHQDPIPKQPSSQPRGDPTGPKE

SEQ ID NO: 21 - DNA, 306 bp, Human immunodeficiency virus
  CDS (1)..(306)
atggaactagtagatcctaacttagatccctggaaccatccaggaagccagcctacaactccttgtaccaaatgctattgtaaaaggtgttgctttcattgccaatggtgctttacaacgaagggcttaggcatctcctatggcaggaagaagcggagacagcgacgaagaactcctcaaagcagtcagatacatcaagatcctgtaccaaagcaacccttatcccaagcccgagggaacccgacaggcccgaaggaatcgaagaaggaggtggagagcaaggcaaagacagatccgtgcgattag

SEQ ID NO: 22 - protein, 101 aa, Human immunodeficiency virus
MELVDPNLDPWNHPGSQPTTPCTKCYCKRCCFHCQWCFTTKGLGISYGRKKRRQRRRTPQSSQIHQDPVPKQPLSQARGNPTGPKESKKEVESKAKTDPCD

SEQ ID NO: 23 - DNA, 306 bp, Human immunodeficiency virus
  CDS (1)..(306)
atggacccggtagatcctaacctagagccctggaatcatccggggagtcagcctaaaactccctgtaacaaatgttattgtaaaatgtgttgctggcattgtcaagtttgctttctgaacaaaggcttaggcatctcctatggcaggaagaagcggaagcaccgacgaggaactcctcagagcagtaaggatcatcaaaatcctgtaccaaagcaacccttacccaccaccagagggaacccgacaggcccgaaggaatcgaagaaggaggtggagagcaagacagagacagatccattcgattag

SEQ ID NO: 24 - protein, 101 aa, Human immunodeficiency virus
MDPVDPNLEPWNHPGSQPKTPCNKCYCKMCCWHCQVCFLNKGLGISYGRKKRKHRRGTPQSSKDHQNPVPKQPLPTTRGNPTGPKESKKEVESKTETDPFD

SEQ ID NO: 25 - DNA, 261 bp, Human immunodeficiency virus
  CDS (1)..(261)
atggacccagtagatcctaaccaagagccctggaaccatccaggaagtcagcctaaaactgcttgtaacaattgttattgtaaaaagtgctgctatcattgccaattgtgctttttaaagaaaggcttaggcatttcctatggcaggaagaagcggagccagcgacgaggaactcctgcaagtttgcaagatcatcaaaatcctataccaaagcaacccttatcccgaacccgcggggacccgacaggcccgaaggaatag

SEQ ID NO: 26 - protein, 86 aa, Human immunodeficiency virus
MDPVDPNQEPWNHPGSQPKTACNNCYCKKCCYHCQLCFLKKGLGISYGRKKRSQRRGTPASLQDHQNPIPKQPLSRTRGDPTGPKE

SEQ ID NO: 27 - DNA, 306 bp, Human immunodeficiency virus
  CDS (1)..(306)
atggagctggtagatcctaacctagagccctggaatcatccgggaagtcagcctacaactgcttgtagcaagtgttactgtaaaatatgttgctggcattgccaactatgctttctgaaaaaaggcttaggcatctcctatggcaggaagaagcggaagcaccgacgaggaactcctcagagcagtaaggatcatcaaaatcctataccagagcaacccctacccatcatcagagggaacccgacagacccgaaagaatcgaagaaggaggtggcgagcaaggcagagacagatccgtgcgattag

SEQ ID NO: 28 - protein, 101 aa, Human immunodeficiency virus
MELVDPNLEPWNHPGSQPTTACSKCYCKICCWHCQLCFLKKGLGISYGRKKRKHRRGTPQSSKDHQNPIPEQPLPIIRGNPTDPKESKKEVASKAETDPCD

SEQ ID NO: 29 - DNA, 306 bp, Human immunodeficiency virus
  CDS (1)..(306)
atggagccggtagatcctagcctagagccctggaaccacccgggaagtcagcctacaactgcttgtagcaattgttactgtaaaatgtgctgctggcattgccaattgtgctttctgaacaagggcttaggcatctcctatggcaggaagaagcggagacgccgacgaggaactcctcagagccgtcaggatcatcaaaatcctgtaccaaagcaacccttacccaccaccagagggaacccgacaggcccgaaagaatcgaagaaggaggtggcgagcaagacagagacagatccgtgcgattag

SEQ ID NO: 30 - protein, 101 aa, Human immunodeficiency virus
MEPVDPSLEPWNHPGSQPTTACSNCYCKMCCWHCQLCFLNKGLGISYGRKKRRRRRGTPQSRQDHQNPVPKQPLPTTRGNPTGPKESKKEVASKTETDPCD

SEQ ID NO: 31 - DNA, 348 bp, Human immunodeficiency virus
  CDS (1)..(348)
atggatccagtagatcctgagatgcccccttggcatcaccctggaagtcagccccagaccccttgtaataagtgctattgcaaaagatgctgctatcattgctatgtttgttttgcaagcaagggtttgggaatctcctatggcaggaagaagcgacggagaccagccgctgctgcgagccatccagataatcaagatcctgtaccagagcaacccccatccatcaccaacaggaagcagaaacgccaggaggaacaggagaaggaggtggagaaggagacaggcccaggtggataccctcgccgcaaggattcttgccactgttgtacacggacctcaggacaataa

SEQ ID NO: 32 - protein, 115 aa, Human immunodeficiency virus
MDPVDPEMPPWHHPGSQPQTPCNKCYCKRCCYHCYVCFASKGLGISYGRKKRRRPAAAASHPDNQDPVPEQPPSITNRKQKRQEEQEKEVEKETGPGGYPRRKDSCHCCTRTSGQ

SEQ ID NO: 33 - protein, 15 aa, Human immunodeficiency virus
MEPVDPRLEPWKHPG

SEQ ID NO: 34 - protein, 15 aa, Human immunodeficiency virus
PRLEPWKHPGSQPKT

SEQ ID NO: 35 - protein, 15 aa, Human immunodeficiency virus
WKHPGSQPKTACTNC

SEQ ID NO: 36 - protein, 15 aa, Human immunodeficiency virus
SQPKTACTNCYCKKC

SEQ ID NO: 37 - protein, 15 aa, Human immunodeficiency virus
ACTNCYCKKCCFHCQ

SEQ ID NO: 38 - protein, 15 aa, Human immunodeficiency virus
YCKKCCFHCQVCFIT

SEQ ID NO: 39 - protein, 15 aa, Human immunodeficiency virus
CFHCQVCFITKALGI

SEQ ID NO: 40 - protein, 15 aa, Human immunodeficiency virus
VCFITKALGISYGRK

SEQ ID NO: 41 - protein, 15 aa, Human immunodeficiency virus
KALGISYGRKKRRQR

SEQ ID NO: 42 - protein, 15 aa, Human immunodeficiency virus
SYGRKKRRQRRRPPQ

SEQ ID NO: 43 - protein, 15 aa, Human immunodeficiency virus
KRRQRRRPPQGSQTH

SEQ ID NO: 44 - protein, 15 aa, Human immunodeficiency virus
RRPPQGSQTHQVSLS

SEQ ID NO: 45 - protein, 15 aa, Human immunodeficiency virus
GSQTHQVSLSKQPTS

SEQ ID NO: 46 - protein, 15 aa, Human immunodeficiency virus
QVSLSKQPTSQSRGD

SEQ ID NO: 47 - protein, 15 aa, Human immunodeficiency virus
KQPTSQSRGDPTGPK

SEQ ID NO: 48 - protein, 15 aa, Human immunodeficiency virus
QSRGDPTGPKEQKKK

SEQ ID NO: 49 - protein, 386 aa, Mus musculus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 50 - protein, 8 aa, Artificial sequence
  Ovalbumin-derived peptide (CFD)
CFDVFKEL

SEQ ID NO: 51 - protein, 8 aa, Artificial sequence
  Ovalbumin-derived peptide (KVV)
KVVRFDKL

SEQ ID NO: 52 - protein, 8 aa, Artificial sequence
  Ovalbumin-derived peptide (SII)
SIINFEKL

SEQ ID NO: 53 - protein, 8 aa, Artificial sequence
  Ovalbumin-derived peptide (OVA1)
ENIFYCPI

SEQ ID NO: 54 - protein, 8 aa, Artificial sequence
  Ovalbumin-derived peptide (OVA2)
AEERYPIL

SEQ ID NO: 55 - protein, 8 aa, Artificial sequence
  Ovalbumin-derived peptide (OVA3)
NAIVFKGL

(19) United States
(12) Patent Application Publication
(10) Pub. No.: US 2007/0275071 A1
(43) Pub. Date: Nov. 29, 2007

(54) Use of Microparticles for Antigen Delivery

(75) Inventors: Barbara Ensoli, I-Roma (IT); Antonella Caputo, San Nicola (IT); Michele Laus, Alessandria (IT); Luisa Tondelli, Bologna (IT); Katia Sparnacci, Coriano (IT); Riccardo Gavioli, Ferrara (IT)

Correspondence Address: CLARK & ELBING LLP, 101 FEDERAL STREET, BOSTON, MA, 02110, US

(73) Assignee: Istituto Superiore Di Sanita, Roma (IT)

(21) Appl. No.: 10/577,974

(22) PCT Filed: Nov. 3, 2004
(86) PCT No.: PCT/EP04/12421

§ 371 Date: May 30, 2007

(30) Foreign Application Priority Data
Nov. 3, 2003 (GB) 0325624.5

(51) Int. Cl. A61K 9/14; A61K 31/74; A61K 38/00; A61P 43/00; A61K 39/00

(52) U.S. Cl. 424/489; 424/188.1; 424/78.31; 514/2

(57) ABSTRACT

The invention relates to microparticles that may be used for antigen delivery and vaccine immunization strategies. The invention in particular relates to microparticles that are useful in the prophylaxis and treatment of human immunodeficiency virus (HIV) infections.

### FIELD OF THE INVENTION

The invention relates to the fields of antigen delivery and vaccines. More specifically, the invention relates to certain microparticles, and to antigen delivery and vaccine immunization strategies employing such microparticles. The invention in particular relates to microparticles that are useful in the prophylaxis and treatment of human immunodeficiency virus (HIV) infections.

### BACKGROUND OF THE INVENTION

It is important that therapeutic or prophylactic peptides, and in particular vaccines, are efficiently delivered to their site of action without significant degradation. Polymeric microparticles encapsulating peptide antigens have been investigated as potential delivery systems for their capability to efficiently target the antigen to professional antigen-presenting cells and to release it in a controlled way over a prolonged period of time (O'Hagan D T., Recent advances in vaccine adjuvants for systemic and mucosal administration, J. Pharm. Pharmacol., 1998; 59:1-10; Nugent J, Wan Po L, Scott E., Design and delivery of non-parental vaccines, Review. J. Clin. Pharm. Therap., 1998; 23:257-85; and Alpar H O, Ward K R, Williamson E D., New strategies in vaccine delivery., S.T.P. Pharma Sci., 2000; 10:269-78).

Although peptides encapsulated into a microparticulate matrix may be protected from unfavorable conditions encountered after parenteral or mucosal administration (Nedrud J G, Lamm M E., Adjuvants and the mucosal immune system, In: Spriggs D R, Koff W C, editors, Topics in vaccine adjuvant research, Boca Raton: CRC, 1991. p. 51 -67), they often become unstable or are degraded. This may occur either during the encapsulation process, such as the exposure to organic solvents, high shear and freeze-drying, and/or in the body when the antigen is exposed to the low pH microenvironment caused by the degradation of the polymer (O'Hagan D T, Singh M, Gupta R K., Poly(lactide-co-glycolide) microparticles for the development of single-dose controlled-release vaccines, Adv. Drug. Deliv. Rev. 1998; 32:22546; and O'Hagan D T., supra).

### SUMMARY OF THE INVENTION

The inventors have found that antigens may be fixed or adsorbed to the external surface of polymeric microparticles. Further the inventors have shown that these microparticles may be used to efficiently deliver antigens to target cells.

Accordingly the invention provides a microparticle comprising:
(a) a core which comprises a water insoluble polymer or copolymer, and
(b) a shell which comprises a hydrophilic polymer or copolymer and functional groups which are ionic or ionisable;
   said microparticle having a disease-associated antigen adsorbed at the external surface.

The invention further provides:
a method of production of a microparticle of invention;
a pharmaceutical composition comprising a microparticle of the invention;
a method of generating an immune response in an individual, said method comprising administering a microparticle of the invention in a therapeutically effective amount;
a method of preventing or treating HIV infection or AIDS, said method comprising administering a microparticle of the invention in a therapeutically effective amount.
a microparticle of the invention for use in a method of treatment of the human or animal body by therapy or diagnosis;
use of a microparticle of the invention for the manufacture of a medicament for generating an immune response in an individual; and
use of a microparticle of the invention for the manufacture of a medicament for preventing or treating HIV infection or AIDS.

### BRIEF DESCRIPTION OF THE FIGURES

FIG. 1 shows BSA (●) and Trypsin (▪) adsorption onto basic (HE1D; A) and acidic (H1D; B) microparticles.

FIG. 2 shows H1D acid microparticles adsorbing the model acid protein β-galactosidase. H1D microparticles were incubated with increasing amounts of protein. H1D/P-galactosidase complexes were centrifuged and supernatants (unbound protein) were collected and analyzed by SDS-PAGE. Pellets (H1D/β-galactosidase complexes) were washed in PBS, and resuspended in 30 ml of NaCl 0.9%, phosphate buffer 5 mM. Samples were boiled for 5 min and spun at 13.000 for 15 min. Supernatants (bound protein) were run onto SDS-PAGE and analyzed by silver staining. Quantification was carried out using a densitometer gel analyzer, as described in materials and methods.

FIG. 3 shows trypsin adsorption on acid microparticles.

FIG. 4 shows BSA adsorption on acid microparticles.

FIG. 5 shows the surface charge density dependence of trypsin adsorption on acid microparticles.

FIG. 6 shows ZP variation of Trypsin/H1D complexes suspended in water.

FIG. 7 shows protein adsorption on H1D acid microparticles.

FIG. 8 shows pH dependance of Trypsin adsorption on acid microparticles (H1D). The amount of trypsin available for adsorption was 50 μg/ml (♦), 150 μg/ml (●) and 300 μg/ml (▪).

FIG. 9 shows trypsin adsorption on acid microparticles (H1D) as a function of buffer ionic strength.

FIG. 10 shows trypsin release from acid microparticles (H1D) in the presence of NaCl and/or SDS. Two separate experiments are shown. The amount of trypsin available for adsorption was 250 μg/ml (A) and 150 μg/ml (B).

FIG. 11 shows analysis of Tat adsorption to the surface of acid polymeric microparticles by FACS analysis using an anti-Tat polyclonal rabbit serum. Two representative microparticles, A7, made of poly(styrene) and hemisuccinated polyvinyl alcohol (●) and 1E, constituted of poly(methyl methacrylate) and Eudragit L100-55 (▪) are shown.

FIG. 12 shows evaluation of cell proliferation in the presence of the microparticles alone or the Tat/microparticle complexes. HL3T1 cells were cultured for 96 h with 10 μg/ml (empty bars), 30 μg/ml (black bars), and 50 μg/ml (gray bars) of microparticles alone (A) or with the same doses of microparticles bound to Tat (1 μg/ml) (B). Controls were represented by untreated cells (None) or cells cultured with 1 μg/ml of Tat (Tat). Results are expressed as the mean (±S.D.) of sextuples.

FIG. 13 shows analysis of in vitro cytotoxicity of 2H1B microparticles. (A) 2H1B, (B) 2H1B /Tat. HL3T1 cells were cultured for 96 hours in the presence of increasing amounts of 2H1B alone (10-500 μg/ml) (left panel) or with the same doses of 2H1B bound to Tat protein (1 μg/ml) (right panel). Controls were represented by untreated cells (none) or cells cultured with Tat alone (1 μg/ml) (Tat). Results are the mean of sextupled wells (±SD).

FIG. 14 shows murine macrophages phagocytosis of polymeric microparticles made of poly(styrene) and hemisuccinated poly(vinyl alcohol) and microparticles made of poly(methyl methacrylate) and Eudragit L100-55. Murine macrophages were cultured with microparticles, fixed, colored with toluidine blue and observed at a phase contrast microscope. Results are expressed as the percentage of cells that phagocytosed the microparticles.

FIG. 15 shows analysis of microparticle uptake. Human monocytes (A), monocyte-derived dendritic cells (B), murine splenocytes (C) and HL3T1 cells (D) were cultured in the presence of fluorescent H1D microparticles for 24 h, fixed with paraformaldheyde and observed at fluorescent and confocal microscopes. Representative images of fluorescent microscopy are shown in panels A, B and C, and of confocal microscopy in panel D.

FIG. 16 shows that polymeric microparticles deliver and release HIV-1 Tat intracellularly. HL3T1 cells were cultured in the presence of fluorescent-H1D (30 μg/ml) bound to Tat (5 μg/ml) (A) or with Tat alone (5 μg/ml) (B), fixed and analyzed by immunofluorescence using an anti-Tat monoclonal antibody. For the same microscopic field, green (H1D), red (Tat), blue (DAPI) and phase contrast (cells) images were taken with a CCD camera and overlapped with a Adobe Photoshop program.

FIG. 17 shows analysis of the expression of the HIV-1 Tat protein bound to polymeric microparticles made of poly(styrene) and hemisuccinated poly(vinyl alcohol) (A4, A7) and of poly(methyl methacrylate) and Eudragit L100-55 (1D, 1E and H1D). HL3T1 cells were incubated with increasing amounts of Tat alone and with the same amounts of Tat bound to each microparticle (30 μg/ml). CAT activity was measured 48 hours later. Results are the mean of three independent experiments.

FIG. 18 shows analysis of the biological activity of Tat bound to 2H1B microparticles. (A) 2H1B/Tat, (B) H1D /Tat; and (C) Tat alone. HL3T1 cells, containing an integrated copy of plasmid HIV-1 -LTR-CAT, where expression of the chloramphenicol acetyl transferase (CAT) reporter gene is driven by the HIV-1 LTR promoter and occurs only in the presence of biologically active Tat, were incubated with increasing amounts of Tat (0.125, 0.5 and 1 μg/ml) bound to 2H1B microparticles (30 μg/ml), or with the same doses of Tat alone, in presence of 100 μM chloroquine. Controls were represented by cells incubated with H1D/Tat complexes (30 μg/ml of H1D and 0.125, 0.5 and 1 μg/ml of Tat) and untreated cells (none). After 48 hours, CAT activity was measured on cell extracts normalized to the protein content. Results are the mean (±SD) of three independent experiments.

FIG. 19 shows analysis of the biological activity of H1D/Tat complexes freshly-made and after lyophilization and storage at room temperature. HL3T1 cells, containing an integrated copy of plasmid HIV-1-LTR-CAT, where expression of the chloramphenicol acetyl transferase (CAT) reporter gene is driven by the HIV-1 LTR promoter and occurs only in the presence of biologically active Tat, were used to test the biological activity of Tat bound to H1D microparticles after lyophilization and storage of the complexes at room temperature. Tat/H1D complexes were prepared, as described in the Examples, using Tat (2 μg/ml) and H1D microparticles (30 μg/ml). Complexes were lyophilized, stored at room temperature for 15 days, resuspended in PBS at room temperature for 1 hour (1 h) or for 4 hours (4 h) and then added to the cells in presence of 100 μM chloroquine. Controls were represented by cells incubated with H1D/Tat complexes prepared and immediately added to the cells (Fresh), Tat alone (Tat) and untreated cells (none). After 48 hours, CAT activity was measured on cell extracts normalized to the protein content.

FIG. 20 shows that polymeric microparticles protect HIV-1 Tat from oxidation. HL3T1 cells, containing an integrated copy of the reporter vector HIV-1 LTR-CAT, were incubated with Tat (1 μg/ml) adsorbed to the microparticles (30 μg/ml) and exposed to air and light for 16 h at room temperature. Control cells were incubated with the same dose of the protein, which was untreated (Tat) or oxidized by exposure to air and light (Tat ox). The percentage of CAT activity was calculated as described (Betti et al., Vaccine, 2001; 19:3408-3419). Results are the mean of two independent experiments.

FIG. 21 shows analysis of the biological activity of Tat/H1D-fluo microparticle complexes freshly-made and after lyophilization and storage at room temperature. HL3T1 cells, containing an integrated copy of plasmid HIV-1-LTR-CAT, where expression of the chloramphenicol acetyl transferase (CAT) reporter gene is driven by the HIV-1 LTR promoter and occurs only in the presence of biologically active Tat, were used to test the biological activity of Tat bound to H1D-fluo microparticles after lyophilization and storage of the complexes at room temperature. Tat/H1D-fluo complexes were prepared, as described in materials and methods, using Tat (2 μg/ml) and H1D-fluo microparticles (30 μg/ml). Complexes were lyophilized, stored at room temperature for 15 days, resuspended in PBS at room temperature for 1 hour (1 h) or for 4 hours (4 h) and then added to the cells in presence of 100 μM chloroquine. Controls were represented by cells incubated with H1D/Tat complexes freshly-prepared (Fresh), Tat alone (Tat) and untreated cells (none). After 48 hours, CAT activity was measured on cell extracts normalized to the protein content.

FIG. 22 shows H1D-fluo microparticles are taken up by cells in vivo and represent a tool for biodistribution studies. Analysis at the site of injection of cellular uptake of H1D-fluorescent microparticles, 15 (panels A and C) and 30 (panels B and D) minutes after inoculation. For the same microscopic field, green (H1D-fluorescent) and blue (nuclei) overlapped images are shown. A, B: 40× magnification; C, D: 100× magnification of images shown in the white square of panels A and B, respectively.

FIGS. 23 shows analysis of γIFN released from splenocytes of mice vaccinated, at weeks 0 and 4, with Tat/microparticle complexes. Splenocytes, obtained two weeks after the second immunization, were pooled by treatment groups, and co-cultured with BALB/c 3T3-Tat expressing cells in the presence of Tat for four days. Results are expressed as pg/ml of γIFN released in culture supernatants.

FIG. 24 shows analysis of T cell proliferation (left panels) and of γIFN release (right panels) in response to Tat-derived 15-mer peptides delivered as A4/Tat (A), H1D/Tat (B.) or just Tat (C). Splenocytes of mice, immunized at weeks 0 and 4 and sacrificed two weeks after the second immunization, were pooled by treatment groups and co-cultured for four days with BALB/c 3T3-Tat expressing cells in the presence of Tat. After Ficoll purification, cells were cultured with irradiated naive splenocytes pulsed with Tat peptides, and with or without PHA. γIFN release on culture supernatants and T-cell ³[H] thymidine incorporation were measured, respectively, after 24 and 96 hours of culture. Only the results to reactive peptides are shown and they are expressed as fold increase of 3[H] thymidine incorporation and release as compared to values of the same cultures grown without PHA.

FIG. 25 shows histologic examination of the inflammatory reactions present at the site of inoculation. Two representative mice received an intramuscular injection with Tat (2 μg) adsorbed to A7 microparticles (A, C) and Tat (2 μg) in Freund's adjuvant (B, D) at weeks 0, 4, and 8. A7-Tat inoculation caused a scarce inflammatory reaction (A) in the muscle fibres consisting exclusively of macrophages (C). Tat plus Freund inoculation induced an intense inflammatory reaction prevalently in the adipose tissue surrounding the muscle fibers with presence of macrophages and clear lacunae of lipolysis (B) and in some cases with extensive necrosis constituted by amorphous material and nuclear debris (D). Hematoxylin-eosin staining; A and B: 40×; C: 400×; and D: 200×.

FIG. 26 shows ovalbumin (acid protein) binding to HE1D basic microparticles. HE1D microspheres were incubated with increasing amounts of ovalbumin. HE1D/ovalbumin complexes were centrifuged and supernatants (unbound protein) were collected and analyzed by SDS-PAGE. Pellets (HE1D/ovalbumin complexes) were washed in PBS, and resuspended in 30 ml of NaCl 0.9%, phosphate buffer 5 mM. Samples were boiled for 5 min and spun at 13.000 for 15 min. Supernatants (bound protein) were run onto SDS-PAGE and analyzed by silver staining. Quantification was carried out using a densitometer gel analyzer, as described in materials and methods.

FIG. 27 shows IgM antibody titers against Tat in vaccinated monkeys.

FIG. 28 shows IgG antibody titers against Tat in vaccinated monkeys.

FIG. 29 shows the lymphoproliferative response of vaccinated monkeys to Tat22 or a pool of Tat peptides.

FIG. 30 shows the results of IFNγ-Elispot assays of vaccinated monkeys in response to Tat22 or a pool of Tat peptides.

### BRIEF DESCRIPTION OF THE SEQUENCE LISTING

SEQ ID NO: 1 shows the nucleotide sequence that encodes the full length. HIV-1 Tat protein from HTLV-III, BH10 CLONE, CLADE B. This is the parent sequence for the TC peptides (SEQ ID NOs: 33 to 48).

SEQ ID NO: 2 shows the 102 amino acid sequence of full length HIV-1 Tat protein from HILV, BH10 CLONE CLADE B.

SEQ ID NOs: 3 to 32 show the nucleotide and amino acid sequences of variants of the full length HIV-1 Tat protein isolated from HTLV-III, BH10 CLONE, CLADE B. The length and sequence of Tat varies depending on the viral isolate.

SEQ ID NO: 3 shows the nucleotide sequence that encodes the shorter version of HIV-1 Tat protein (BHH10).

SEQ ID NO: 4 shows the 86 amino acid shorter version of HIV-1 Tat protein (BH10). This sequence corresponds to residues 1 to 86 of SEQ ID NO: 1.

SEQ ID NO: 5 shows the nucleotide sequence that encodes the cysteine 22 mutant of BH10 (SEQ ID NO: 4).

SEQ ID NO: 6 shows the 86 amino acid cysteine 22 mutant of BH1O (SEQ ID NO: 4).

SEQ ID NO: 7 shows the nucleotide sequence that encodes the lysine 41 mutant of BH10 (SEQ ID NO: 4).

SEQ ID NO: 8 shows the 86 amino acid lysine 41 mutant of BH10 (SEQ ID NO: 4).

SEQ ID NO: 9 shows the nucleotide sequence that encodes the RGDA mutant of BH10 (SEQ ID NO: 4).

SEQ ID NO: 10 shows the 83 amino acid RGDA mutant of BH10 (SEQ ID NO: 4).

SEQ ID NO: 11 shows the nucleotide sequence that encodes the lysine 41 RGDA mutant of BH10 (SEQ ID NO: 4).

SEQ ID NO: 12 shows the 83 amino acid lysine 41 RGDA mutant of BH10 (SEQ ID NO: 4).

SEQ ID NO: 13 shows the nucleotide sequence that encodes the consensus_A-A1-A2 variant of HIV-1 Tat protein.

SEQ ID NO: 14 shows the 101 amino acid consensus_A-A1-A2 variant of HIV-1 Tat protein.

SEQ ID NO: 15 shows the nucleotide sequence that encodes the consensus_B variant of HIV-1 Tat protein.

SEQ ID NO: 16 shows the 101 amino acid consensus_B variant of HIV-1 Tat protein.

SEQ ID NO: 17 shows the nucleotide sequence that encodes the consensus_C variant of HIV-1 Tat protein.

SEQ ID NO: 18 shows the 101 amino acid consensus_C variant of HIV-1 Tat protein.

SEQ ID NO: 19 shows the nucleotide sequence that encodes the consensus_D variant D of HIV-1 Tat protein.

SEQ ID NO: 20 shows the 86 amino acid consensus_D variant of the HIV-1 Tat protein.

SEQ ID NO: 21 shows the nucleotide sequence that encodes the consensus_F1-F2 variant of HIV-1 Tat protein.

SEQ ID NO: 22 shows the 101 amino acid consensus_F1-F2 variant of HIV-1 Tat protein.

SEQ ID NO: 23 shows the nucleotide sequence that encodes the consensus_G variant of the HIV-1 Tat protein.

SEQ ID NO: 24 shows the 101 amino acid consensus_G variant of the HIV-1 Tat protein.

SEQ ID NO: 25 shows the nucleotide sequence that encodes the consensus_H variant of the HIV-1 Tat protein.

SEQ ID NO: 26 shows the 86 amino acid consensus_H variant of the HIV-1 Tat protein.

SEQ ID NO: 27 shows the nucleotide sequence that encodes the consensus_CRF01 variant of the HIV-1 Tat protein.

SEQ ID NO: 28 shows the 101 amino acid consensus_CRF01 variant of the HIV-1 Tat protein.

SEQ ID NO: 29 shows the nucleotide sequence that encodes the consensus_CRF02 variant of the HIV-1 Tat protein.

SEQ ID NO: 30 shows the 101 amino acid consensus_CRF02 of the HIV-1 Tat protein.

SEQ ID NO: 31 shows the nucleotide sequence that encodes the consensus_O variant of HIV-1 Tat protein.

SEQ ID NO: 32 shows the 115 amino acid consensus_O variant of the HIV-1 Tat protein.

SEQ ID NO: 33 shows the sequence of the TC27 peptide in Table 8.

SEQ ID NO: 34 shows the sequence of the TC28 peptide in Table 8.

SEQ ID NO: 35 shows the sequence of the TC29 peptide in Table 8.

SEQ ID NO: 36 shows the sequence of the TC30 peptide in Table 8.

SEQ ID NO: 37 shows the sequence of the TC31 peptide in Table 8.

SEQ ID NO: 38 shows the sequence of the TC32 peptide in Table 8.

SEQ ID NO: 39 shows the sequence of the TC33 peptide in Table 8.

SEQ ID NO: 40 shows the sequence of the TC34 peptide in Table 8.

SEQ ID NO: 41 shows the sequence of the TC35 peptide in Table 8.

SEQ ID NO: 42 shows the sequence of the TC36 peptide in Table 8.

SEQ ID NO: 43 shows the sequence of the TC37 peptide in Table 8.

SEQ ID NO: 44 shows the sequence of the TC38 peptide in Table 8.

SEQ ID NO: 45 shows the sequence of the TC39 peptide in Table 8.

SEQ ID NO: 46 shows the sequence of the TC40 peptide in Table 8.

SEQ ID NO: 47 shows the sequence of the TC41 peptide in Table 8.

SEQ ID NO: 48 shows the sequence of the TC42 peptide in Table 8.

SEQ ID NO: 49 shows the sequence of Ovalbumin adsorbed onto HE1D microparticles.

SEQ ID NO: 50 shows the sequence of the CFD peptide in Table 11.

SEQ ID NO: 51 shows the sequence of the KVV peptide in Table 11.

SEQ ID NO: 52 shows the sequence of the SII peptide in Table 11.

SEQ ID NO: 53 shows the sequence of the OVA1 peptide in Table 11.

SEQ ID NO: 54 shows the sequence of the OVA2 peptide in Table 11.

SEQ ID NO: 55 shows the sequence of the OVA3 peptide in Table 11.

### DETAILED DESCRIPTION OF THE INVENTION

It is to be understood that this invention is not limited to particular antigens. It is also to be understood that different applications of the disclosed methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an antigen” includes a mixture of two or more such agents, reference to “a microparticle” includes reference to mixtures of two or more microparticles, reference to “a target” cell” includes two or more such cells, and the like.

All publications, patents and patent applications cited herein, whether supra or infra, are hereby incorporated by reference in their entirety.

The invention provides microparticles for delivering antigens to target cells. The microparticles have an antigen adsorbed or fixed onto their external surface. The term “microparticle of the invention” is herein defined as a microparticle with an antigen adsorbed at the external surface.

The microparticles comprise: a core which comprises a water insoluble polymer or copolymer; and a shell which comprises a hydrophilic polymer or copolymer and functional groups which are ionic or ionisable. The microparticles are typically obtainable by dispersion polymerization of a water-insoluble monomer in the presence of a hydrophilic polymer or copolymer. The water-insoluble monomer is polymerized to form the core and the hydrophilic polymer or copolymer forms the shell. The outer shell is typically covalently bonded to the inner core. The external microparticle surface is typically a hydrophilic shell that comprises ionic or ionisable chemical groups. The microparticle surface has an overall positive or negative charge. The microparticles are cationic or anionic. The microparticles preferably have a net positive or negative charge over their entire external surface. The surface charge density typically varies across the surface of the microparticles.

The shell and core of the microparticles are preferably composed of a biocompatible polymeric material. The term “biocompatible polymeric material” is defined as a polymeric material which is not toxic to an animal and not carcinogenic. The matrix material may also be biodegradable in the sense that the polymeric material should degrade by bodily processes in vivo to products readily disposable by the body and should not accumulate in the body. On the other hand, where the microparticle is being inserted into a tissue which is naturally shed by the organism (eg. sloughing of the skin), the matrix material need not be biodegradable.

Suitable water insoluble polymer forming materials for use in the core of the microparticles include, but are not limited to, poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. The polymeric materials may be cross-linked.

Preferred materials include, but are not limited to, polyacrylates, polymethacrylates and polystyrenes. The term “poly(meth)acrylate” as used herein encompasses both polyacrylates and polymethacrylates. Likewise the term “(meth)acrylate” encompasses both acrylates and methacrylates.

Preferred poly(meth)acrylates which may be used as core materials include poly(alkyl (meth)acrylates), in particular poly(C₁₋₆ alkyl (meth)acrylates), and preferably poly(C₁₋₆ alkyl (meth)acrylates) such as poly(methyl acrylate), poly(methyl methacrylate), poly(ethyl acrylate), and poly(ethyl methacrylate). Poly(methyl methacrylate) (PMMA) is especially preferred as the core material.

Suitable hydrophilic polymer forming materials for use in the hydrophilic shell of the microparticles include, but are not limited to, hemisuccinated polyvinylalcohols and Eudragit® copolymers.

A preferred material for the hydrophilic shell is a polymer or copolymer which comprises repeating units of formula I: 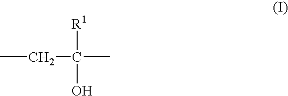
wherein R1 is hydrogen, methyl or ethyl.

The hydrophilicity may be augmented by reacting this polymer with a diacid such as maleic or succinic acid. A particularly preferred hydrophilic polymer is hemisuccinated polyvinylalcohol.

Another preferred class of hydrophilic polymer that may be used in the hydrophilic shell of the microparticles is a copolymer which comprises repeating units of formulae (II) and (III): 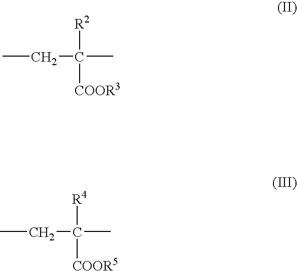
wherein R² and R⁴ each independently represent hydrogen or methyl, R³ represents hydrogen, -A-NR⁹R¹⁰ or -A-N⁺R⁹R¹⁰R¹¹X⁻, in which A represents C₁₋₁₀ alkylene, R⁹, R¹⁰ and R¹¹ each independently represent hydrogen or C₁₋₁₀ alkyl and X represents halogen, and R⁵ represents C₁₋₁₀ alkyl.

In a particular embodiment, R² in the repeating unit of formula (II) is hydrogen or methyl.

In a particular embodiment, R³ in the monomer of formula (II) represents hydrogen or -A-NR⁹R¹⁰

A in the monomer of formula (II) is C₁₋₁₀ alkylene and is preferably a C₁₋₆ alkylene group, for example a methylene, ethylene, propylene, butylene, pentylene or hexylene group or isomer thereof. Ethylene is preferred.

R⁹ in the monomer of formula (II) is hydrogen or C₁₋₁₀ alkyl, and is preferably a C₁₋₁₀ alkyl group, more preferably a C₁₋₆ alkyl group, for example a methyl, ethyl, propyl, i-propyl, -butyl, sec-butyl or tert-butyl group, or a pentyl or hexyl group or isomer thereof Methyl and ethyl are preferred, particularly methyl.

R¹⁰ in the monomer of formula (I) is hydrogen or C₁₋₁₀ alkyl, and is preferably a C₁₋₁₀ alkyl group, more preferably a C₁₋₆ alkyl group, for example a methyl, ethyl, propyl, i-propyl, n-butyl, sec-butyl or tert-butyl group, or a pentyl or hexyl group or isomer thereof. Methyl and ethyl are preferred, particularly methyl.

R⁴ in the repeating unit of formula (III) is hydrogen or methyl.

R⁵ in the repeating unit of formula (III) is C₁₋₁₀ alkyl, and is preferably a C₁₋₆ alkyl group, for example a methyl, ethyl, propyl, i-propyl, n-butyl, sec-butyl or tert-butyl group, or a pentyl or hexyl group or isomer thereof. Methyl, ethyl and butyl are preferred.

An example of a copolymer comprising repeating units of formulae (II) and (III) which may be used in the present invention is a copolymer of methacrylic acid and ethyl acrylate, for example a statistical copolymer in which the ratio of the free carboxyl groups to the ester groups is approximately 1:1. A suitable copolymer is commercially available from Röhm Pharma under the trade name Eudragit® L 100-55.

A further example of a copolymer comprising repeating units of formulae (II) and (III) which may be used in the present invention is a copolymer of 2-(dimethylamino)ethyl methacrylate and C₁₋₆ alkyl methacrylate, for example a copolymer of 2-(dimethylamino)ethyl methacrylate, methyl methacrylate and butyl methacrylate. A suitable copolymer is commercially available from Röhm Pharma under the trade name Eudragit® E 100.

The hydrophilic polymer forming materials contain chemical groups that are ionic or ionisable. Preferably these groups are ionic or ionisable at physiological pH. The term “physiological pH” refers to the pH in the blood and extracellular fluid of an individual. The physiological pH is typically from 7.2 to 7.6 and preferably 7.4.

These water insoluble and hydrophilic polymeric materials may be used alone, as physical mixtures (blends) or as copolymers (which may be block copolymers). Again, these polymers may be cross-linked. The copolymers may be block, random or regular copolymers.

Usually, a satisfactory number-average molecular weight is in the range of 5,000 to 500,000 daltons, more preferably in the range of 10,000 to 500,000 daltons. The polymers mentioned above generally have number-average molecular weights of from 30,000 to 50,000 daltons, up to about 120,000 daltons such as from 80,000 to 100,000 daltons. A person skilled in the art would understand the appropriate number-average molecular weight range for a specific polymer.

Conventional methods for the construction of microparticles may be used to construct the microparticles of the invention. The microparticles are obtainable by dispersion polymerization of monomers. This method is described in Sparnacci et al. Macromolecular Chemistry and Physics, 2002: 203 (10-11): 1364-1369. Polymers are formed by the polymerization of one monomer. Copolymers are formed by the polymerization of more than one monomer. Thus one or more water insoluble core monomers may be included in the polymerization reaction. Thus one or more hydrophilic shell polymers may be included in the polymerization reaction.

Typically, the core monomer, shell polymer and a radical initiator are dissolved in a suitable solvent under a nitrogen atmosphere. Suitable solvents include organic solvents such as acetone, halogenated hydrocarbons such as chloroform, methylene chloride and the like, aromatic hydrocarbon compounds, halogenated aromatic hydrocarbon compounds, cyclic ethers, alcohols, ethyl acetate and the like. Preferred solvents are methanol, ethanol, a 1:1 ratio mixture of ethanol and 2-methoxyethanol and a mixture of methanol and water (in a ratio between 7:3 and 9:1). The mixture of materials in the solvent may undergo freeze thaw cycles depending on the polymeric materials used. The temperature during the formation of the dispersion is not especially critical but can influence the size and quality of the microparticles. Moreover, depending on the solvent employed, the temperature must not be too low or the solvent and processing medium will solidify or the processing medium will become too viscous for practical purposes, or too high that the processing medium will evaporate, or that the liquid processing medium will not be maintained. Accordingly, the dispersion process can be conducted at any temperature which maintains stable operating conditions, which preferred temperature being about 30° C. to 80° C., depending upon the materials selected.

The dispersed microparticles may be isolated from the solvent by any convenient means of separation. Thus, for example, the reaction mixture may undergo several rounds of centrifugation and redispersion with the solvent followed by several rounds of centrifugation and redispersion in water.

Following the isolation of the microparticles from the dispersion solvent, the microparticles may be dried by exposure to air or by other conventional drying techniques such as lyophilization, vacuum drying, drying over a desiccant, or the like. Prior to absorption the microparticles may be redispersed in a suitable liquid and temporarily stored. The skilled person will recognise under what conditions the microparticles of the invention may be stored. Typically, the microparticles are stored at a low temperature, for example 4° C.

The microparticles usually have a spherical shape, although irregularly-shaped microparticles are possible. When viewed under a microscope, therefore, the particles are typically spheroidal but may be elliptical, irregular in shape or toroidal. The microparticles vary in size, ranging from 0.1 μm to 10 μm, typically from 0.5 μm or 0.75 μm to 4 μm, or typically from 1 μm, 1.5 μm or 2.5 μm to 6 μm. The maximum size is the diameter in spherical microparticles.

The size of the microparticles can be measured using conventional techniques such as microscopic techniques (where particles are sized directly and individually rather than grouped statistically), absorption of gasses, or permeability techniques. If desired, automatic particle-size counters can be used (for example, the Coulter Counter, HIAC Counter, or Gelman Automatic Particle Counter) to ascertain average particle size.

Actual microparticle density can be readily ascertained using known quantification techniques such as helium pycnometry and the like. Alternatively, envelope (“tap”) density measurements can be used to assess the density of a particulate composition. Envelope density information is particularly useful in characterizing the density of objects of irregular size and shape. Envelope density, or “bulk density,” is the mass of an object divided by its volume, where the volume includes that of its pores and small cavities. Other, indirect methods are available which correlate to density of individual particles. A number of methods of determining envelope density are known in the art, including wax immersion, mercury displacement, water absorption and apparent specific gravity techniques. A number of suitable devices are also available for determining envelope density, for example, the GeoPyc™ Model 1360, available from the Micromeritics Instrument Corp. The difference between the absolute density and envelope density of a sample pharmaceutical composition provides information about the sample's percentage total porosity and specific pore volume.

Microparticle morphology, particularly the shape of a particle, can be readily assessed using standard light or electron microscopy. It is preferred that the particles have a substantially spherical or at least substantially spherical shape. It is also preferred that the particles have an axis ratio of 2 or less, i.e. from 2:1 to 1:1, to avoid the presence of rod- or needle-shaped particles. These same microscopic techniques can also be used to assess the particle surface characteristics, for example, the amount and extent of surface voids or degree of porosity.

In an especially preferred embodiment, the microparticles comprise a core of poly(styrene) and a hydrophilic shell of hemisuccinated poly(vinyl alcohol) and have an average size of from 0.9 μm to 4 μm. In another especially preferred embodiment, the microparticles comprise a core of poly(methyl methacrylate) and a hydrophilic shell of Eudragit® E100 and have a average size from 1.5 μm to 8.5 μm. In a further especially preferred embodiment, the microparticles comprise a core of poly(methyl methacrylate) and a hydrophilic shell of Eudragit® L100/55 and have an average size from 1.5 μm to 2.0 μm.

The term “adsorbed” or “fixed” means that the microbial antigen is attached to the external surface of the shell of the microparticle. The absorption or fixation preferably occurs by electrostatic attraction. Electrostatic attraction is the attraction or bonding generated between two or more ionic or ionisable chemical groups which are oppositely charged. The absorption or fixation is typically reversible.

The antigen preferably has a net charge that attracts it to the ionic hydrophilic shell of the microparticle. The antigen typically has one or more charged chemical or ionic groups. In the case of the antigen being a peptide, the antigen typically has one or more charged amino acid residues. The antigen typically has a net positive or negative charge. The antigen preferably has a net charge that is opposite to the charge of the hydrophilic shell of the microparticle. As a result, basic antigens may be adsorbed onto acid microparticles and acidic antigens may be adsorbed onto basic microparticles.

The antigen may be adsorbed onto the microparticles by mixing a solution of the antigen with a liquid suspension of the microparticles. The antigen and microparticles are typically mixed in a suitable liquid, for example a physiological buffer such as phosphate buffered saline (PBS). The mixture may be left for sometime under conditions suitable for the preservation of the antigen and formation of the bond between the antigen and microparticles. These conditions will be recognised by a person skilled in the art. Adsorption is preferably carried out at 0° to 37° C., preferably 4 to 25° C. and in the dark. Adsorption is typically carried out for from 30 and 180 minutes. Following adsorption, the microparticles of the invention may be separated from the adsorption liquid by methods known in the art, for example centrifugation. The microparticle-antigen complexes may then be resuspended in a liquid suitable for administration to an individual.

The term “disease-associated antigen” is used in it broadest sense to refer to any antigen associated with a disease. An antigen is a molecule which contains one or more epitopes that will stimulate a host's immune system to make a cellular antigen-specific immune response, and/or a humoral antibody response. Thus, a disease-associated antigen is a molecule which contains epitopes that will stimulate a host's immune system to make a cellular antigen-specific immune response and/or a humoral antibody response against the disease. The disease-associated antigen may therefore be used for prophylactic or therapeutic purposes.

Disease-associated antigens are preferably associated with infection by microbes, typically microbial antigens, or associated with cancer, typically tumours. Thus, antigens that may be used in the invention include proteins, polypeptides, immunogenic protein fragments, oligosaccharides, polysaccharides, and the like. The term “immunogenic fragment” means a fragment of any antigen described herein that itself is capable of stimulating a host's immune system to make a cellular antigen-specific immune response and/or a humoral antibody response.

The disease-associated antigen may be associated with microbial infection and thus contain epitopes that will stimulate a host's immune system to make a cellular antigen-specific immune response and/or a humoral antibody response against the microbial infection. The antigen is typically found in the body of an individual when that individual has a microbial infection. The antigen is preferably derived from a microbe, namely microbial. Thus, the antigen may be derived from any known microbe, for example, virus, bacterium, parasites, protists such as protozoans, or fungus, and can be a whole organism or immunogenic parts thereof, for example, cell wall components.

Antigens for use in the invention include, but are not limited to, those containing, or derived from, members of the families Picomaviridae (for example, polioviruses, etc.); Caliciviridae; Togaviridae (for example, rubella virus, dengue virus, etc.); Flaviviridae; Coronaviridae; Reoviridae; Birnaviridae; Rhabodoviridae (for example, rabies virus, measels virus, respiratory syncytial virus, etc.); Orthomyxoviridae (for example, influenza virus types A, B and C, etc.); Bunyaviridae; Arenaviridae; Retroviradae (for example, HTLV-I; HTLV-II; HIV-1; and HIV-2); simian immunodeficiency virus (SIV) among others. Additionally, viral antigens may be derived from a papilloma virus (for example, HPV); a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV) and the tick-borne encephalitis viruses; smallpox, parainfluenza, varicella-zoster, cytomeglavirus, Epstein-Barr, rotavirus, rhinovirus, adenovirus, papillomavirus, poliovirus, mumps, rubella, coxsackieviruses, equine encephalitis, Japanese encephalitis, yellow fever, Rift Valley fever, lymphocytic choriomeningitis, and the like. See for example, Virology, 3^{rd} Edition (W. K. Joklik ed. 1988); Fundamental Virology, 2^{nd} Edition (B. N. Fields and D. M. Knipe, eds. 1991), for a description of these and other viruses.

Bacterial antigens include, but are not limited to, those containing or derived from organisms that cause diphtheria, cholera, tuberculosis, tetanus, pertussis, meningitis, and other pathogenic states, including Meningococcus A, B and C, Hemophilus influenza type B (HIB), and Helicobacter pylori, Streptococcus pneumoniae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidun, Leptspirosis interrogans, Borrelia burgdorferi, Campylobacter jejuni, and the like.

Examples of anti-parasitic antigens include, but are not limited to, those derived from organisms causing malaria and Lyrne disease. Antigens of such fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like.

In a especially preferred embodiment, the antigen adsorbed on the microparticle is the HIV Tat protein (SEQ ID NO: 2, 4, 6, 8, 10, 12, 14, 16, 18, 20, 22, 24, 26, 28, 30 or 32) or an immunogenic fragment thereof.

The disease-associated antigen may be cancer-associated. A cancer-associated antigen is a molecule which contains epitopes that will stimulate a host's immune system to make a cellular antigen-specific immune response and/or a humoral antibody response against the cancer. A cancer-associated antigen is typically found in the body of an individual when that individual has cancer. A cancer-associated antigen is preferably derived from a tumor. Cancer-associated antigens include, but are not limited to, cancer-associated antigens (CAA), for example, CAA-breast, CAA-ovarian and CAA-pancreatic; the melanocyte differentiation antigens, for example, Melan A/MART-1, tyrosinase and gp100; cancer-genn cell (CG) antigens, for example, MAGE and NY-ESO-1; mutational antigens, for example, MUM-1, p53 and CDK4; over-expressed self-antigens, for example, p53 and HER2/NEU and tumor proteins derived from non-primary open reading frame mRNA sequences, for example, LAGE1.

Synthetic antigens are also included in the definition of antigen, for example, haptens, polyepitopes, flanking epitopes, and other recombinant or recombinant or synthetically derived antigens (Bergmann et al. (1993) Eur. J. Immunol. 23:2777-2781; Bergmann et al. (1996) J. Immunol. 157:3242-3249; Suhrbier, A. (1997) Immunol. and Cell Biol. 75:402408; Gardner et al. (1998) 12^{th} World AIDS Conference, Geneva, Switzerland (Jun. 28-Jul. 3, 1998). A synthetic disease-associated antigen is a synthetic molecule which contains epitopes that will stimulate a host's immune system to make a cellular antigen-specific immune response and/or a humoral antibody response against the disease.

The antigen or immunogenic fragments of antigens mentioned herein typically comprise one or more T cell epitopes. “T cell epitopes” are generally those features of a peptide structure capable of inducing a T cell response. In this regard, it is accepted in the art that T cell epitopes comprise linear peptide determinants that assume extended conformations within the peptide-binding cleft of MHC molecules (Unanue et al. (1987) Science 236: 551-557). As used herein, a T cell epitope is generally a peptide having about 8-15, preferably 5-10 or more amino acid residues.

The microparticles of the invention can be viewed as a “vaccine composition” and as such includes any pharmaceutical composition which contains an antigen and which can be used to prevent or treat a disease or condition in a subject. The term encompasses both subunit vaccines, i.e., vaccine compositions containing antigens which are separate and discrete from a whole organism with which the antigen is associated in nature, as well as compositions containing whole killed, attenuated or inactivated bacteria, viruses, parasites or other microbes. The vaccine can also comprise a cytokine that may further improve the effectiveness of the vaccine.

The microparticles of the invention can comprise from about 1 to about 99% of the antigen by weight, for example from about 0.01 to about 10% of the antigen by weight. The microparticles can therefore comprise from 0.05 to 10% of the antigen by weight such as from 2 to 8% of the antigen by weight or from 5 to 6% of the antigen by weight. The actual amount depends on a number of factors include the nature of the antigen, the dose desired and other variables readily appreciated by those skilled in the art.

The inventors have shown that administration of microparticles of the invention generates an immune response in an individual. Thus the inventors have shown that adsorption of the antigen to the external surface of the microparticle preserves the biological activity of the antigen. Thus the inventors have also shown that the adsorption of the antigen to the microparticle does not affect the immunogenicity of the antigen. The inventors have also shown that adsorption of the antigen to the microparticle reduce the amount of antigen required to generate an immune response, eliminates or reduces the number of antigen booster shots needed and improves the handling or shelf-life of the antigen.

Accordingly, the present invention also relates to prophylactic or therapeutic methods utilising the microparticles of the invention. These prophylactic or therapeutic methods involve generating an immune response in an individual using the microparticles of the invention. Thus, the microparticles of the invention may be administered to an individual to generate an immune response in that individual. Alternatively, the microparticles may be used in the manufacture of a medicament for generating an immune response in an individual.

The term “administer” or “deliver” is intended to refer to any delivery method of contacting the microparticles with the target cells or tissue. The term “tissue” refers to the soft tissues of an animal, patient, subject etc. as defined herein, which term includes, but is not limited to, skin, mucosal tissue (eg. buccal, conjunctival, gums), vaginal and the like. Bone may however be treated too by the particles of the invention, for example bone fractures.

When administration is for the purpose of treatment, administration may be either for prophylactic or therapeutic purpose. When provided prophylactically, the antigen is provided in advance of any symptom. The prophylactic administration of the antigen serves to prevent or attenuate any subsequent symptom. When provided therapeutically the antigen is provided at (or shortly after) the onset of a symptom. The therapeutic administration of the antigen serves to attenuate any actual symptom. Administration and therefore the methods of the invention may be carried out in vivo or in vitro.

The terms “animal”, “individual”, “patient” and “subject” are used interchangeably herein to refer to a subset of organisms which include any member of the subphylum cordata, including, without limitation, humans and other primates, including non-human primates such as chimpanzees and other apes and monkey species; farm animals such as bovine animals, for example cattle; ovine animals, for example sheep; porcine, for example pigs; rabbit, goats and horses; domestic mammals such as dogs and cats; wild animals; laboratory animals including rodents such as mice, rats and guinea pigs; birds, including domestic, wild and game birds such as chickens, turkeys and other gallinaceous birds, ducks, geese; and the like. The terms do not denote a particular age. Thus, both adult and newborn individuals are intended to be covered. In one embodiment, the individual is typically capable of being infected by HIV.

The invention includes treating a disease state in an animal by administering the microparticles described herein to a subject in need of such treatment. As used herein, the term “treatment” or “teating” includes any of the following: the prevention of infection or reinfection; the reduction or elimination of symptoms; and the reduction or complete elimination of a pathogen. Treatment may be effected prophylactically (prior to infection) or therapeutically (following infection). The methods of this invention also include effecting a change in an organism by administering the microparticles.

The methods of the invention may be carried out on individuals at risk of disease associated with antigen. Typically, the methods of the invention are carried out on individuals at risk of microbial infection or cancer associated with or caused by the antigen. In a preferred embodiment, the method of the invention is carried out on individuals at risk of infection with HIV or developing AIDS.

The methods described herein elicit an immune response against particular antigens for the treatment and/or prevention of a disease and/or any condition which is caused by or exacerbated by the disease. The methods described herein typically elicit an immune response against particular antigens for the treatment and/or prevention of microbial infection or cancer and/or any condition which is caused by or exacerbated by microbial infection or cancer. In a particular embodiment, the methods described herein elicit an immune response against particular antigens for the treatment and/or prevention of HIV infection and/or any condition which is caused by or exacerbated by HIV infection, such as AIDS.

The method of the invention is carried out for the purpose of stimulating a suitable immune response. By suitable immune response, it is meant that the method can bring about in an immunized subject an immune response characterized by the increased production of antibodies and/or production of B and/or T lymphocytes specific for an antigen, wherein the immune response can protect the subject against subsequent infection. In a preferred embodiment, the method can bring about in an immunized subject an immune response characterized by the increased production of antibodies and/or production of B and/or T lymphocytes specific for HIV-1 Tat, wherein the immune response can protect the subject against subsequent infection with homologous or heterologous strains of HIV, reduce viral burden, bring about resolution of infection in a shorter amount of time relative to a non-inmunized subject, or prevent or reduce clinical manifestation of disease symptoms, such as AIDS symptoms.

The aim of the method of the invention is to generate an immune response in an individual. Preferably, antibodies to the antigen are generated in the individual. Preferably IgG antibodies to the antigen are generated. Antibody responses may be measured using standard assays such as radioimmunoassay, ELISAs and the like, well known in the art.

Preferably cell-mediated immunity is generated, and in particular a CD8 T cell response generated. In this case the administration of the microparticles may, for example increases the level of antigen experienced CD8 T cells. The CD8 T cell response may be measured using any suitable assay (and thus may be capable of being detected in such an assay), such as an ELISPOT assay, preferably an γIFN-ELISPOT assay, CD8 proliferation to peptides and CTL assays. Preferably, a CD4 T cell response is also generated, such as the CD4 Th1 response. Thus the levels of antigen experienced CD4 T cells may also be increased. Such increased levels of CD4 T cells may be detected using a suitable assay, such as a proliferation assay.

The invention further provides the microparticles of the invention, namely microparticles with adsorbed antigens, in a pharmaceutical composition which also includes a pharmaceutically acceptable excipient. Such an “excipient” generally refers to a substantially inert material that is nontoxic and does not interact with other components of the composition in a deleterious manner.

These excipients, vehicles and auxiliary substances are generally pharmaceutical agents that do not themselves induce an immune response in the individual receiving the composition, and which may be administered without undue toxicity.

Pharmaceutically acceptable excipients include, but are not limited to, liquids such as water, saline, polyethyleneglycol, hyaluronic acid, glycerol and ethanol. Pharmaceutically acceptable salts can be included therein, for example, mineral acid salts such as hydrochlorides, hydrobromides, phosphates, sulphates, and the like; and the salts of organic acids such as acetates, propionates, malonates, benzoates, and the like.

It is also preferred, although not required, that an antigen composition will contain a pharmaceutically acceptable carrier that serves as a stabilizer, particularly for peptide, protein or other like antigens. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. It may also be useful to employ a charged lipid and/or detergent. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, for example, TWEEN® surfactants (Atlas Chemical Industries, Wilmington, Del.), polyoxyethylene ethers, for example Brij, pharmaceutically acceptable fatty acid esters, for example, lauryl sulfate and salts thereof (SDS), and like materials.

A thorough discussion of pharmaceutically acceptable excipients, carriers, stabilizers and other auxiliary substances is available in REMINGTONS PHARMACEUTICAL SCIENCES (Mack Pub. Co., New Jersey 1991), incorporated herein by reference.

In order to augment an immune response in a subject, the compositions and methods described herein can further include ancillary substances/adjuvants as well as the compound of the invention, such as pharmacological agents, cytokines, or the like. Suitable adjuvants include any substance that enhances the immune response of the subject to the antigens attached to the microparticles of the invention. They may enhance the immune response by affecting any number of pathways, for example, by stabilizing the antigen/MHC complex, by causing more antigen/MHC complex to be present on the cell surface, by enhancing maturation of APCs, or by prolonging the life of APCs (e. g., inhibiting apoptosis).

Typically adjuvants are co-administered with the vaccine or rnicroparticle. As used herein the term “adjuvant” refers to any material that enhances the action of a antigen or the like.

Thus, one example of an adjuvant is a “cytokine”. As used herein, the term “cytokine” refers to any one of the numerous factors that exert a variety of effects on cells, for example, inducing growth, proliferation or maturation. Certain cytokines, for example TRANCE, flt-3L, and CD40L, enhance the inumunostimulatory capacity of APCs. Non-limiting examples of cytokines which may be used alone or in combination include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1 a), interleukin-11 (IL-11), MIP-1a, leukemia inhibitory factor (LIF), c-kit ligand, thrombopoietin (TPO), CD40 ligand (CD40L), tumor necrosis factor-related activation-induced cytokine (TRANCE) and flt3 ligand (flt-3L). Cytokines are commercially available from several vendors such as, for example, Genzyme (Framingham, Mass.), Genentech (South San Francisco, Calif.), Amgen (Thousand Oaks, Calif.), R & D Systems and Immunex (Seattle, Wash.).

The sequence of many of these molecules are also available, for example, from the GenBank database. It is intended, although not always explicitly stated, that molecules having similar biological activity as wild-type or purified cytokines (for example, recombinantly produced or mutants thereof) and nucleic acid encoding these molecules are intended to be used within the spirit and scope of the invention.

A composition which contains the microparticles of the invention and an adjuvant, or a vaccine or microparticles of the invention which is co-administered with an adjuvant, displays “enhanced immunogenicity” when it possesses a greater capacity to elicit an immune response than the immune response elicited by an equivalent amount of the vaccine administered without the adjuvant. Such enhanced immunogenicity can be determined by administering the adjuvant composition and microparticle controls to animals and comparing antibody titers and/or cellular-mediated immunity between the two using standard assays such as radioimmunoassay, ELISAs, CTL assays, and the like, well known in the art.

In the method of the invention the microparticles of the invention are typically delivered in liquid form or delivered in powdered form. Liquids containing the microparticles of the invention are typically suspensions. The microparticles of the invention may be administered in a liquid acceptable for delivery into an individual. Typically the liquid is a sterile buffer, for example sterile phosphate-buffered saline (PBS).

The microparticles of the invention are typically delivered parenterally, either subcutaneously, intravenously, intramuscularly, intrastemally or by infusion techniques. A physician will be able to determine the required route of administration for each particular patient.

The vaccine or microparticles are typically delivered transdermally. The term “transdermal” delivery intends intradermal (for example, into the dermis or epidermis), transdermal (for example,“percutaneous”) and transmucosal administration, for example, delivery by passage of an agent into or through skin or mucosal (for example buccal, conjunctival or gum) tissue. See, for example, Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987).

Delivery may be via conventional needle and syringe for the liquid suspensions containing microparticle particulate. In addition, various liquid jet injectors are known in the art and may be employed to administer the microparticles. Methods of determining the most effective means and dosages of administration are well known to those of skill in the art and will vary with the delivery vehicle, the composition of the therapy, the target cells, and the subject being treated. Single and multiple administrations can be carried out with the dose level and pattern being selected by the attending physician. The liquid compositions are administered to the subject to be treated in a manner compatible with the dosage formulation, and in an amount that will be prophylactically and/or therapeutically effective.

The microparticles themselves in particulate composition (for example, powder) can also be delivered transdermally to vertebrate tissue using a suitable transdermal particle delivery technique. Various particle delivery devices suitable for administering the substance of interest are known in the art, and will find use in the practice of the invention. A transdermal particle delivery system typically employs a needleless syringe to fire solid particles in controlled doses into and through intact skin and tissue. Various particle delivery devices suitable for particle-mediated delivery techniques are known in the art, and are all suited for use in the practice of the invention. Current device designs employ an explosive, electric or gaseous discharge to propel the coated core carrier particles toward target cells. The coated particles can themselves be releasably attached to a movable carrier sheet, or removably attached to a surface along which a gas stream passes, lifting the particles from the surface and accelerating them toward the target. See, for example, U.S. Pat. No. 5,630,796 which describes a needleless syringe. Other needleless syringe configurations are known in the art.

Delivery of particles from such particle delivery devices is practiced with particles having an approximate size generally ranging from 0.1 to 250 μm. The actual distance which the delivered particles will penetrate a target surface depends upon particle size (e. g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm3, preferably between about 0.9 and 1.5 g/cm3, and injection velocities generally range between about 100 and 3,000 m/sec, or greater. With appropriate gas pressure, particles having an average diameter of 10-70 um can be accelerated through the nozzle at velocities approaching the supersonic speeds of a driving gas flow.

The powdered compositions are administered to the subject to be treated in a manner compatible with the dosage formulation, and in an amount that will be prophylactically and/or therapeutically effective.

Microparticles comprising prophylactically or therapeutically effective amount of the antigen described herein can be delivered to any suitable target tissue via the above-described particle delivery devices. For example, the compositions can be delivered to muscle, skin, brain, lung, liver, spleen, bone marrow, thymus, heart, lymph, blood, bone cartilage, pancreas, kidney, gall bladder, stomach, intestine, testis, ovary, uterus, rectum, nervous system, eye, gland and connective tissues.

A “therapeutically effective amount” is defined very broadly as that amount needed to give the desired biologic or pharmacologic effect. This amount will vary with the relative activity of the antigen to be delivered and can be readily determined through clinical testing based on known activities of the antigen being delivered. The “Physicians Desk Reference” and “Goodman and Gilman's The Pharmacological Basis of Therapeutics” are useful for the purpose of determined the amount needed in the case of known pharmaceutical agents. The amount of microparticles administered depends on the organism( for example animal species), antigen, route of administration, length of time of treatment and, in the case of animals, the weight, age and health of the animal. One skilled in the art is well aware of the dosages required to treat a particular animal with an antigen.

Commonly, the microparticles are administered in microgram amounts. The coated microparticles are administered to the subject to be treated in a manner compatible with the dosage formulation, and in an amount that will be effective to bring about a desired immune response. The amount of the microparticles to be delivered which, is 1 μg to 5 mg, more typically 1 to 50, μg of peptide, depends on the subject to be treated. The exact amount necessary will vary depending on the age and general condition of the individual being immunized and the particular nucleotide sequence or peptide selected, as well as other factors. An appropriate effective amount can be readily determined by one of skill in the art upon reading the instant specification.

Mixed populations of different types of microparticles can be combined into single dosage forms and can be co-administered. The same antigen can be incorporated into the different microparticle types that are combined in the final formulation or co-administered. Thus, multiphasic delivery of the same antigen can be achieved. Alternatively, different antigens may be adsorbed onto the different microparticle types combined in a formulation. For example, a formulation may comprise a negatively charged antigen adsorbed to positively charged microparticles and a positively charged antigen adsorbed to negatively charged microparticles. Different antigens may therefore be co-administered in a single dosage form.

Below are examples of specific embodiments for carrying out the present invention. The examples are offered for illustrative purposes only, and are not intended to limit the scope of the present invention in any way.

### EXAMPLES

1. Microparticles

Reagents

Benzoyl peroxide (BPO), polyvinylalcohol (molar mass 49000), styrene, succinic anydride, methyl methacrylate were purchased from Aldrich. Methacrylic acid/ethylacrylate 1/1 (mol/mol) statistical copolymer (trade name Eudragit® L100-55) was supplied by Röhm Pharma as a powder sample and is characterized by a number-average molar mass of 250000 g/mol. Butyl methacrylate/2-dimethylamino ethyl methacrylate/methyl methacrylate 1/2/1 (mol/mol) copolymer (trade name Eudragit® E100) was supplied by Röhm Pharma as a powder sample and is characterized by a number-average molar mass of 150000 g/mol. BSA and Bradford Reagent were purchased from Sigma. Methanol (99,9%, Carlo Erba) and 2,2′-azobis (isobutyronitrile) (AIBN) (98.0%, Fluka) were used without further purification. Methyl methacrylate (MMA) (99%, Aldrich) was distilled under vacuum immediately before use.

Synthesis of Fluorescent Monomer

2.0 g of fluoresceine (6.0 mmol), 2.0 g of calcium carbonate and hydroquinone (trace) were dissolved in 100 ml of DMF, and the solution was heated at 60° C. Allyl chloride was added slowly dropwise and the reaction was allowed to proceed for 30 h in the dark. After vacuum evaporation of the solvent the product was purified by flash column chromatography (silica gel; diethyl ether-ethyl acetate 80:20 as eluent). Yield 53%, (m.p.=123-125° C.); MS, m/z (%): 412 (M+, 100), 371 (10), 287 (20), 259 (15), 202 (7); ¹H-NMR (CD₃OD): δ 4.44 (dd, J=5.9 and 1 Hz, 2 H, O—CH₂—CH═), 4.75 (dd, J=5.9 and 1 Hz, 2 H, O—CH₂—CH═), 5.08 (m, 2H, CH₂═CH), 5.40 (m, 2H, CH₂═CH), 5.58 (m, 1H, CH₂═CH), 6.10 (m, 1H, CH₂═CH), 6.60 (m, 2H, Ar), 6.98 (m, 3H, Ar), 7.25 (d, J=1 Hz, 1H, Ar), 7.45 (dd, J=7.5 and 1 Hz, 1H, Ar), 7.85 (m, 2H, Ar), 8.30 (dd, J=7.5 and 1 Hz, 1H, Ar).

Acid Microparticles

Microparticles A4 and A7 were prepared by dispersion polymerization of styrene (monomer) in the presence of hemisuccinated poly(vinyl alcohol) as the steric stabilizer. Microparticles 1D, 1E, H1D and fluorescent H1D were obtained by dispersion polymerization of methyl methacrylate (monomer) in the presence of Eudragit® L100-55 as the steric stabilizer. The microparticles were produced by dispersion polymerization. The physico-chemical properties of these mircoparticles are described in Table 1 below.

As a typical example, the preparation of the microparticle sample A7 (polystyrene and hemisuccinated polyvinylalcohol) was as follows: 1.86 g of hemisuccinated polivinylalcohol, 15.5 ml of styrene, 1.95 g of BPO were dissolved in 162 ml of ethanol/2-methoxyethanol 1/1 under a nitrogen atmosphere; three freeze-thaw cycles were run. A4 microparticles were prepared with a similar procedure starting from 1.34 g of hemisuccinated polyvinylalcohol dissolved in 162 ml of ethanol/2-methoxyethanol 9/1. The solution was heated at 78° C. for 48 hours under mechanic stirring (60 rpm). The reaction mixture was then cooled and, after three cycles of centrifugation and redispersion with the organic solvent and two cycles with HPLC grade water, the resulting particles were lyophilized. The resulting yields were 76% and 82% respectively.

As a typical example for the Eudragit stabilized polymethylmethacrylate microparticles, the preparation of the sample 1D is described: 14.73 g of Eudragit® L100-55 was dissolved under a nitrogen atmosphere for 30 min in 200 ml methanol heated at 60° C. A 0.37 g portion of 2,2′-azobis (isobutyronitrile) (AIBN) was dissolved in 18.4 g of methylmethacrylate monomer and added to solution. 1E microparticles were prepared in a similar way starting from 18.10 g of Eudragit. The reaction was left to proceed for 24 hrs under constant stiring. The reaction mixture was then cooled and, after three cycles of centrifugation and redispersion with methanol and then two cycles with HPLC grade water, the resulting particles were lyophilized. The resulting yields were 78% and 65% respectively.

As a further typical example for the Eudragit stabilized polymethylmethacrylate microparticles, the preparation of sample H1D is described: 7.36 g of Eudragit® L 100-55 were dissolved in 200 ml of a solution of methanol/water 76/24 wt-% and heated at 60° C. with mechanical stirring (speed of stirring 300 g/min) under nitrogen atmosphere and reflux condenser. After 30 min, 370 mg (2.25 mmol) of AIBN, dissolved in 18.3 g (183 mmol) of methyl methacrylate, were added to the solution and the reaction was allowed to proceed for 24 h. At the end of the reaction, the latex was cooled and then purified by four cycles of centrifugation (2000 g/min for 10 minutes) and redispersion with methanol and HPLC grade water. The reaction yield was 76.2%, as determined gravimetrically. Fluorescent H1D was obtained by reacting fluorescent monomer (see above) with together methyl methacrylate in the dispersion reaction. 11.0 g of Eudragit® L 100-55 was dissolved in 200 ml of a solution of methanol water 76/24 wt-% and heated at 60° C. with mechanical stirring (speed of stirring 300 g/min) under nitrogen atmosphere and reflux condenser. After 30 min, 370 mg (2.25 mmol) of MBN and 5.0 mg (12.1 μmol) of fluorescent monomer, dissolved in 18.3 g (183 mmol) of methyl methacrylate, were added to the solution and the reaction was allowed to proceed for 24 h. At the end of the reaction, the microparticles were purified as previously described. The experimental conditions for the preparation of the acid microparticles is shown in Table 1.

TABLE 1
Experimental conditions^{a,b,c} for the preparation
of acid microparticle samples.

| | MeOH | H₂O | MMA | AIBN | Eud. L100-55 | Yield |
|---|---|---|---|---|---|---|
| Sample | wt.- % | wt.- % | wt. % | wt.- % | Wt.- % | % |
| 1A | 87.8 | / | 10.0 | 0.2 | 2.0 | 64.2 |
| 1B | 86.0 | / | 10.0 | 0.2 | 3.8 | 76.2 |
| 1C | 83.9 | / | 10.0 | 0.2 | 5.9 | 69.5 |
| 1D | 82.0 | / | 10.0 | 0.2 | 7.8 | 70.1 |
| 1E | 80.0 | / | 10.0 | 0.2 | 9.8 | 65.3 |
| H1A | 66.7 | 21.1 | 10.0 | 0.2 | 2.0 | 67.5 |
| H1B | 65.4 | 20.6 | 10.0 | 0.2 | 3.8 | 64.6 |
| H1C | 63.8 | 20.1 | 10.0 | 0.2 | 5.9 | 73.0 |
| H1D | 62.3 | 19.7 | 10.0 | 0.2 | 7.8 | 78.6 |
| H1E | 59.9 | 19.1 | 10.0 | 0.2 | 9.8 | 73.1 |
| 1H1C | 63.9 | 20.2 | 10.0 | 0.05 | 5.9 | 56.6 |
| 2H1C | 63.8 | 20.2 | 10.0 | 0.1 | 5.9 | 60.1 |
| 3H1C | 63.7 | 20.1 | 10.0 | 0.3 | 5.9 | 80.5 |
| 1H1B | 46.8 | 39.4 | 10.0 | 0.05 | 3.8 | 74.8 |
| 2H1B | 46.8 | 39.3 | 10.0 | 0.1 | 3.8 | 86.7 |
| 3H1B | 46.7 | 39.3 | 10.0 | 0.2 | 3.8 | 88.1 |
| H1Dfluo | 63.8 | 20.1 | 10.0 | 0.2 | 5.9 | 66.3 |

^{a}Based on total recipe (184.0 g).
^{b}The dispersion polymerization reactions were performed at 60° C. for 24 h under continuous stirring, nitrogen atmosphere and a reflux condenser.
^{c}For samples H1A-H1E, 1H1C-3H1C and H1D fluo, the ratio between methanol and water in the solvent mixture is 76/24 wt.-%, whereas for samples 1H1B-3H1B is 54/46 wt.-%.
^{d}For sample H1D fluo, the reaction was performed in presence of 5.0 mg of fluoresceine derivative 3.

Basic Microparticles

Microparticles HE1D (diameter 0.48 μm±0.03) were prepared by dispersion polymerization of methyl methacrylate (monomer) in the presence of Eudragit® E100 as the steric stabilizer. 14.73 g of Eudragit were dissolved in 200 ml of a solution of methanol/water 76/24 wt-% and heated at 60° C. with mechanical stirring (speed stirring 300 g/min) under nitrogen atmosphere and reflux condenser. After 30 min, 370 mg (2.25 mmol) of AIBN dissolved in 18.3 g (183 mmol) of MMA were added to the solution and the reaction was allowed to proceed for 24 hr. At the end of the reaction the microparticles were purified as previously described.

TABLE 2
Experimental conditions^{a,b,c} for the preparation
of basic microparticle samples.

| | MeOH | H₂O | MMA | AIBN | Eudragit E 100 | Yield |
|---|---|---|---|---|---|---|
| Sample | wt.- % | wt.- % | wt. % | wt.- % | wt.- % | % |
| E1Z | 88.8 | / | 10.0 | 0.2 | 1.0 | 77.8 |
| E1A | 87.8 | / | 10.0 | 0.2 | 2.0 | 62.3 |
| E1B | 86.0 | / | 10.0 | 0.2 | 3.8 | 82.0 |
| E1C | 83.9 | / | 10.0 | 0.2 | 5.9 | 61.6 |
| E1D | 82.0 | / | 10.0 | 0.2 | 7.8 | 70.8 |
| HE1Z | 67.5 | 21.3 | 10.0 | 0.2 | 1.0 | 63.4 |
| HE1A | 66.7 | 21.1 | 10.0 | 0.2 | 2.0 | 64.6 |
| HE1B | 65.4 | 20.6 | 10.0 | 0.2 | 3.8 | 66.5 |
| HE1C | 63.8 | 20.1 | 10.0 | 0.2 | 5.9 | 79.5 |
| HE1D | 62.3 | 19.7 | 10.0 | 0.2 | 7.8 | 74.3 |
| 0.5E1B | 86.3 | / | 10.0 | 0.05 | 3.8 | 59.2 |
| 1E1B | 86.1 | / | 10.0 | 0.1 | 3.8 | 65.1 |
| 2E1B | 86.0 | / | 10.0 | 0.2 | 3.8 | 82.0 |
| 3E1B | 85.9 | / | 10.0 | 0.3 | 3.8 | 83.2 |

^{a}Based on total recipe (183.5 g).
^{b}For samples HE1Z-HE1D the ratio between methanol and water in the solvent mixture is 76/24 wt.-%.
^{c}The dispersion polymerization reactions were performed at 60° C. for 24 h under continuous mechanical stirring, nitrogen atmosphere and a reflux condenser.

Physico-chemical Characterization

i) Morphological characterization: particle size and size distribution were measured using a JEOL JSM-35CF scanning electron microscope (SEM) operating at an accelerating voltage of 20 kV. The samples were sputter coated under vacuum with a thin layer (10-30 Å) of gold. The SEM photographs were digitalizated and elaborated by the Scion Image processing program. From 200 to 250 individual microparticle diameters were measured for each sample.

ii) Determination of amount of steric stabilizer on the external surface of the microparticles: for acidic microparticles, the amount of steric stabilizer linked to the microparticle surface was determined by back titration of the excess NaOH after complete salification of the acid groups and microparticle removal by centrifugation. The salification was accomplished by dispersing in a beaker 0.6 g of a microparticle sample in 10 ml of 20 mM NaOH at room temperature for 24 h. Then, the microparticle sample was removed by centrifugation and washed twice with 25 ml of distilled water. The supernatants were combined and the excess NaOH was titrated with 20 mM HCl.

For basic microparticles, the amount of steric stabilizer was determined by back titration of the excess HCl after complete salification of the aminic groups and microparticles removal. The salification was accomplished by dispersing in a beaker 0.6 g of a microparticle sample in 10 ml of 20 mM HCl at room temperature. The microparticles were removed by centrifugation and washed twice with water. The supernatants were combined and the excess HCl was titrated with 20 mM NaOH.

The physico-chemical properties of the acidic microparticles are shown in Tables 3 and 4 below. The physico-chernical properties of the basic microparticles are shown in Table 5 below.

TABLE 3
Acid microparticles physico-chemical characterization

| | | Surface | Surface |
|---|---|---|---|
| | SEM diameter | charge density | charge density |
| Sample | (μm) | (μmol/g) | (μmol/m²) |
| A4 | 0.99 ± 0.03 | 8.1 | 72.5 |
| A7 | 3.46 ± 0.10 | 4.6 | 30.9 |
| 1D | 4.35 ± 1.02 | 48.1 | 37.8 |
| 1E | 2.60 ± 0.45 | 59.2 | 27.3 |
| H1D | 1.69 ± 0.16 | 62.1 | 17.8 |
| H1Dfluo | 2.13 ± 0.09 | 59.2 | 21.1 |

TABLE 4
Number average diameter ( D̅ₙ), weight average diameter ( D̅_{w}),
uniformity ratio (U), amount of acid groups per gram of microparticles
and the surface charge density for samples.

| | | | | COOH/ | COOH/ |
|---|---|---|---|---|---|
| | D̅ₙ | D̅_{w} | | microparticle | microparticle |
| Sample | μm | μm | U | μmol/g | nmol/cm² |
| 1A | 10.18 | 11.03 | 1.08 | 20.6 | 3.69 |
| 1B | 6.15 | 6.54 | 1.06 | 22.0 | 2.35 |
| 1C | 2.49 | 5.38 | 2.16 | 29.0 | 2.01 |
| 1D | 4.35 | 4.80 | 1.10 | 48.1 | 3.78 |
| 1E | 2.60 | 2.28 | 1.11 | 59.2 | 2.73 |
| H1A | 2.42 | 3.89 | 1.61 | 27.2 | 1.42 |
| H1B | 2.38 | 2.49 | 1.05 | 42.7 | 1.75 |
| H1C | 2.36 | 2.45 | 1.04 | 54.2 | 2.13 |
| H1D | 1.69 | 1.73 | 1.02 | 62.1 | 1.78 |
| H1E | 1.77 | 1.83 | 1.04 | 65.3 | 1.98 |
| 1H1C | 1.64 | 1.68 | 1.02 | 65.4 | 1.34 |
| 2H1C | 2.17 | 2.22 | 1.02 | 58.9 | 1.94 |
| 3H1C | 2.40 | 2.41 | 1.01 | 55.9 | 2.24 |
| 1H1B | 0.80 | 0.72 | 1.03 | 68.7 | 0.95 |
| 2H1B | 0.65 | 0.67 | 1.03 | 60.2 | 0.65 |
| 3H1B | 0.78 | 0.80 | 1.02 | 57.3 | 0.75 |
| H1Dfluo | 2.13 | 2.14 | 1.01 | 59.2 | 2.11 |

TABLE 5
Number average diameter ( D̅ₙ), weigth average diameter ( D̅_{w}),
uniformity ratio (U) and amount of amino groups per gram
of basic microparticle samples.

| | | | | NR₂/ |
|---|---|---|---|---|
| | D̅ₙ | D̅_{w} | | microparticle |
| Sample | μm | μm | U | mol/g · 10⁻⁶ |
| E1Z | 4.70 | 5.50 | 1.17 | 3.57 |
| E1A | 2.52 | 4.03 | 1.60 | 6.94 |
| E1B | 1.70 | 1.90 | 1.12 | 19.5 |
| E1C | 0.93 | 1.23 | 1.32 | 29.9 |
| E1D | 1.24 | 1.29 | 1.04 | 33.9 |
| HE1Z | 0.96 | 1.28 | 1.33 | 6.99 |
| HE1A | 0.79 | 0.82 | 1.04 | 12.6 |
| HE1B | 1.12 | 1.14 | 1.02 | 10.5 |
| HE1C | 0.99 | 1.05 | 1.06 | 26.0 |
| HE1D | 0.48 | 0.53 | 1.10 | 28.1 |
| 0.5E1B | 1.02 | 1.59 | 1.56 | 29.9 |
| 1E1B | 1.27 | 1.28 | 1.01 | 23.2 |
| 2E1B | 1.70 | 1.90 | 1.12 | 19.5 |
| 3E1B | 2.04 | 2.05 | 1.01 | 7.61 |

2. Protein Adsorption and Release Experiments in Cell-free Systems

Tables 6 and 7 show the acidic and basic microparticles investigated.

TABLE 6
Acid microparticles (Eudragit L100-55)
investigated in cell-free systems

| | | | ζ- | Surface | Surface | Surface |
|---|---|---|---|---|---|---|
| | SEM | PCS | potential | area | charge | charge |
| Sample | (μm) | (μm) | (mV) | (m²/g) | (μmol/g) | (μmol/m²) |
| H1D | 1.69 | n.d. | −52.4 | 3.50 | 62.1 | 17.8 |
| H1D fluo | 2.13 | n.d. | −53.9 | 2.81 | 59.2 | 21.1 |
| 1H1B | 0.80 | 1.039 | −47.7 | 7.23 | 68.7 | 9.5 |
| 2H1B | 0.63 | 0.857 | −49.8 | 9.26 | 60.2 | 6.5 |

TABLE 7
Basic microparticles (Eudragit E100)
investigated in cell-free systems

| | | | ζ- | Area | Surface | Surface |
|---|---|---|---|---|---|---|
| | SEM | PCS | potential | superficial | charge | charge |
| Sample | (μm) | (μm) | (mV) | (m²/g) | (μmol/g) | (μmol/m²) |
| HE1A | 0.79 | 1.037 | +54.7 | 7.38 | 12.6 | 1.71 |
| HE1B | 1.12 | n.d. | n.d. | 5.28 | 10.5 | 1.99 |
| HE1C | 0.99 | 1.026 | +59.6 | 5.80 | 26.0 | 4.48 |
| HE1D | 0.480 | 0.801 | +35.4 | 11.7 | 28.1 | 2.40 |

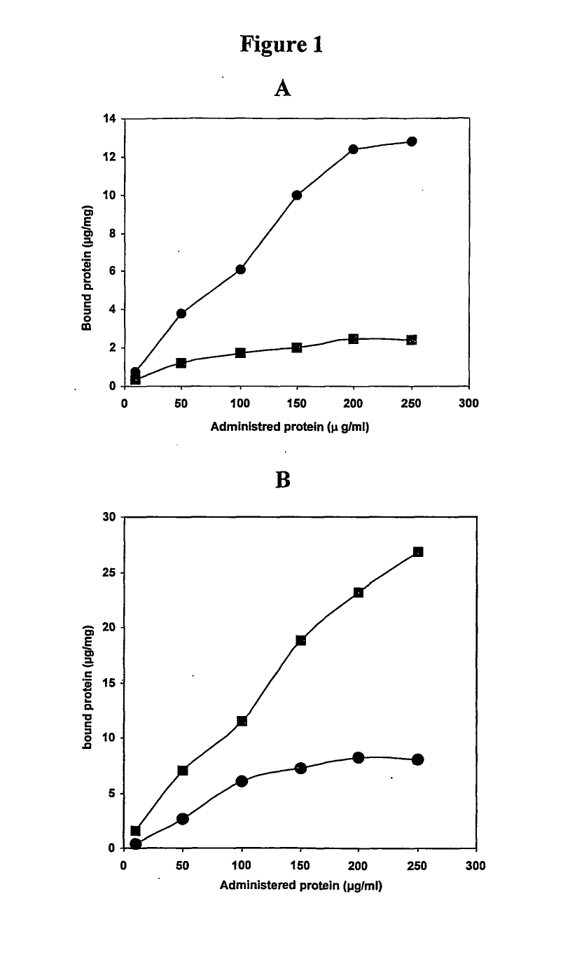

As a typical example, the adsorption behaviour of BSA (66.432 Kda, pI=5.46) and trypsin (23.783 Kda, pI=9.64), as model proteins, was investigated on the H1D and HE1D samples. 5.0 mg of H1D or HE1D was incubated in 1.0 ml of a 20 mM sodium phosphate buffer solution at pH 7.4 in the presence of different concentrations of protein (from 10 to 250 μg/ml) for 2 h. Then, the microparticle sample was removed by centrifugation at 15000 g/min for 10 min and the amount of the residual protein on the supernatant was estimated using the Bradford colorimetric method (Bradford, M. M. Anal. Biochem. 1976, 72, 248) or the Bicinchoninic Assay (BCA). The amount of adsorbed BSA or trypsin was then calculated as the difference between the feed and the residual BSA or trypsin. Experiments were run in triplicate. (SD<10%). For release experiments, the pellets were washed twice with water and then left 2 hours under stirring at room temperature in the presence of 1M NaCl phosphate buffer (pH 7,4). The amount of released protein was determined by UV/VIS absorbance. Cell-free binding experiments with BSA and Trypsin show that the amount of adsorbed protein increased as the protein concentration increased which suggested a high compatibility of protein toward the microparticle surface (see FIG. 1).

To determine whether acid H1D microparticles adsorb acid proteins, β-galactosidase (β-gal) was chosen as the model protein. β-galactosidase was purchased from Roche (cat. 567779; Penzberg, Germany). Its molecular weight and isolectric point are 116.000 Daltons and 5.28, respectively. The protein was resuspended (2 mg/ml) in water and stored at 4° C.

H1D/β-gal complexes were prepared in PBS with 0.5, 1, 2, 5 and 10 μg of β-gal protein and 30 μg of H1D microparticles (70 μl final volume). After 1 hour incubation, complexes were collected by centrifigation at 13.000 rpm for 10 min. Supernatants (unbound protein) were collected and analyzed by SDS-polyacrylamide gel electrophoresis (PAGE). Pellets (H1D/β-gal complexes) were washed twice in PBS, and resuspended in 30 μl of NaCl 0.9%, phosphate buffer 5 mM. Samples were boiled for 5 min and spun at 13.000 for 15 min. Supernatants (bound protein) were run onto 14% SDS-PAGEs and analyzed by silver staining (Davis L G, Dibner M D, Battey J F. In: Davis L G, Dibner M D, Battey J F, editors. Basic Methods in Molecular Biology. New York: Elsevier, 1986). Quantification was carried out using a densitometer gel analyzer (Quantity-One, BioRad Laboratories, Milan, Italy) as compared to known amounts of β-gal (0.5, 1, 2 and 5 μg) migrated in each gel. The percentage of protein bound to the particle surface was determined as: 100× adsorbed protein (μg)/administered protein (μg).

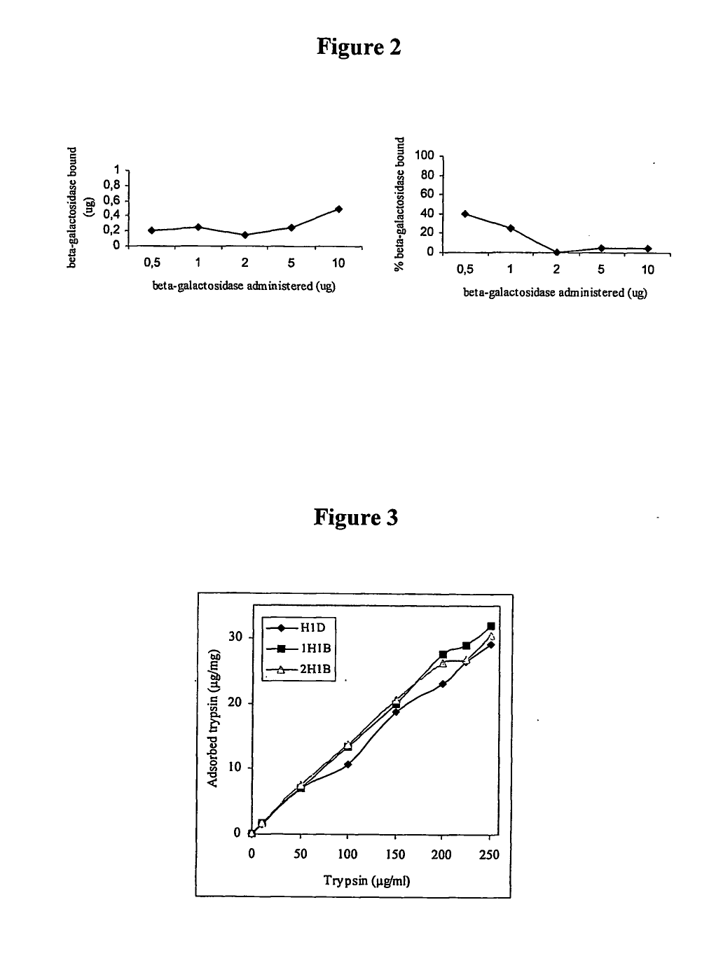

Thus, increasing doses of β-gal were incubated with H1D to allow adsorption, and the amount of protein adsorbed onto the microparticles surface was analysed by SDS-PAGE, as described in more detail above. The results indicated that β-gal adsorbs at the surface of these acid microparticles (FIG. 2A). The adsorption efficiency resulted inversely correlated with the dose of administered protein, being higher (40%) with the lower dose (0.5 μg) of β-gal (FIG. 2B).

These results indicate that H1D acid microparticles can bind also acid proteins, although with a lower efficiency as compared to the binding efficiency of basic proteins (i.e. HIV-1 Tat, trypsin). These results confirm the results of different binding cell-free experiments with H1D and another acid protein, BSA, described above.

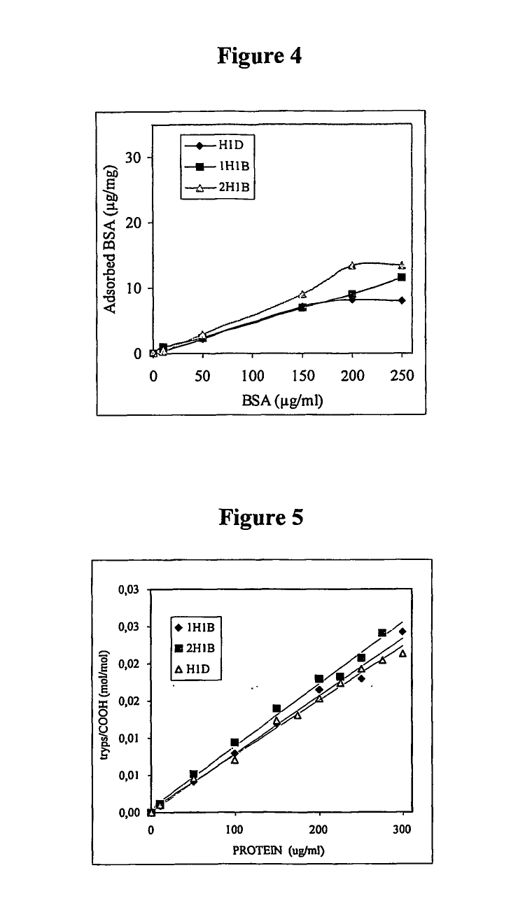

In order to establish if acid microparticles are able to establish strong ionic interactions with basic proteins, adsorption experiments with trypsin (pI=9.64) were run at physiological pH too. Despite their differences in size and surface charge density, all the microparticle samples were able to adsorb trypsin on their surface with high efficiency rate (54-81%) in a wide concentration range (0-300 μg/ml), reaching high loading values up to 3% w/w (FIG. 3). In a parallel experiment, a model acid protein (BSA) was adsorbed with lower efficiency rates (13-48%), thus reaching lower loading values (FIG. 4).

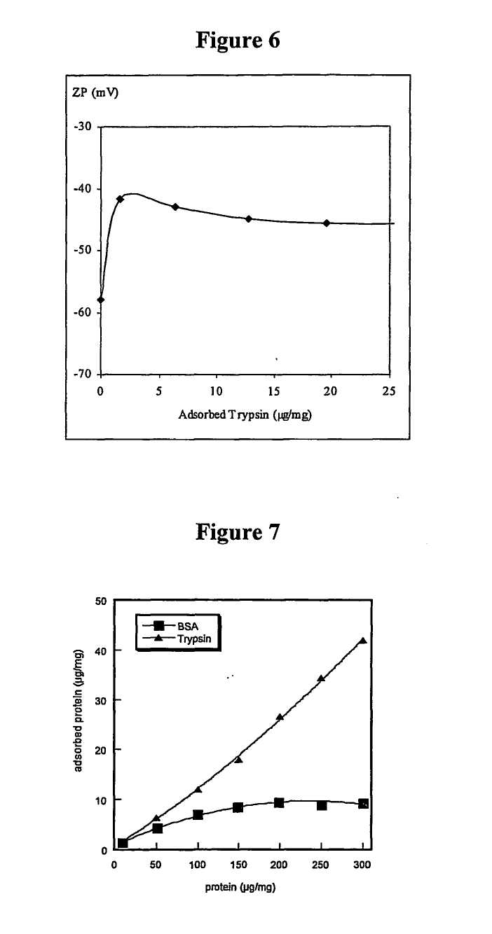

Trypsin adsorption on acid microparticles is mainly driven by ionic interaction with carboxylic groups deriving from Eudragit L100-55 chains covalently bound to the particle surface (FIG. 5). On the contrary, BSA adsorption fail to correlate with particle surface charge density. Electrophoretic mobility variations of microparticle sample H1D as a function of adsorbed trypsin was measured by means of dynamic light scattering techniques, showing a reduction of zeta potential values (ZP) while increasing the surface coverage degree (FIG. 6). Binding/release experiments were run as a function of protein concentration as well as of buffer pH and ionic strength. A new calorimetric method was employed (BCA instead of Bradford) due to its higher reproducibility and sensitivity. Acid microparticles H1D show higher adsorption rates for basic proteins (i.e. trypsin) with respect to acid proteins (i.e. BSA) (FIG. 7).

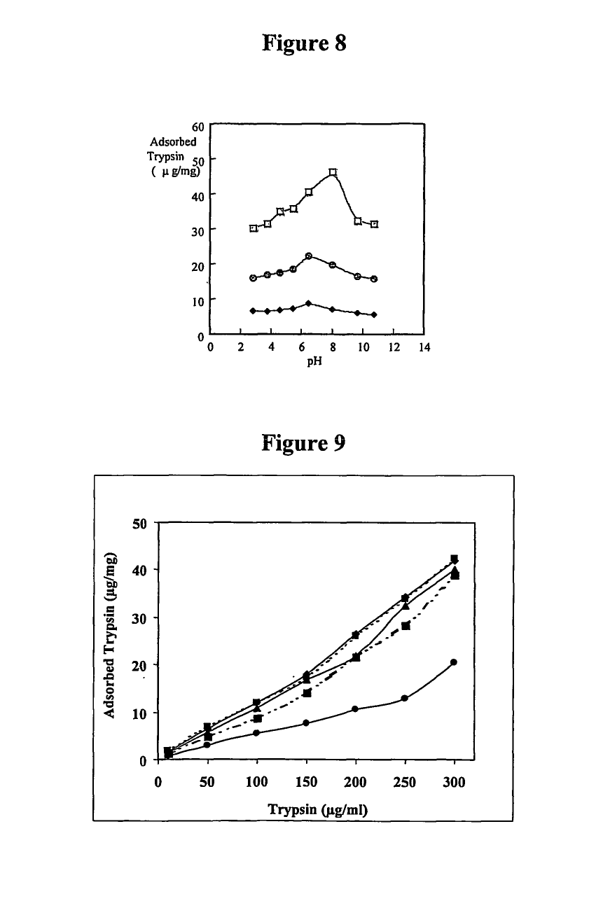

Trypsin adsorption on acid microparticles H1D is greatly reduced in the presence of acid and basic buffers (FIG. 8) as well as in the presence of high salt concentration (FIG. 9) thus confirming the main ionic nature of trypsin interaction with the particle surface.

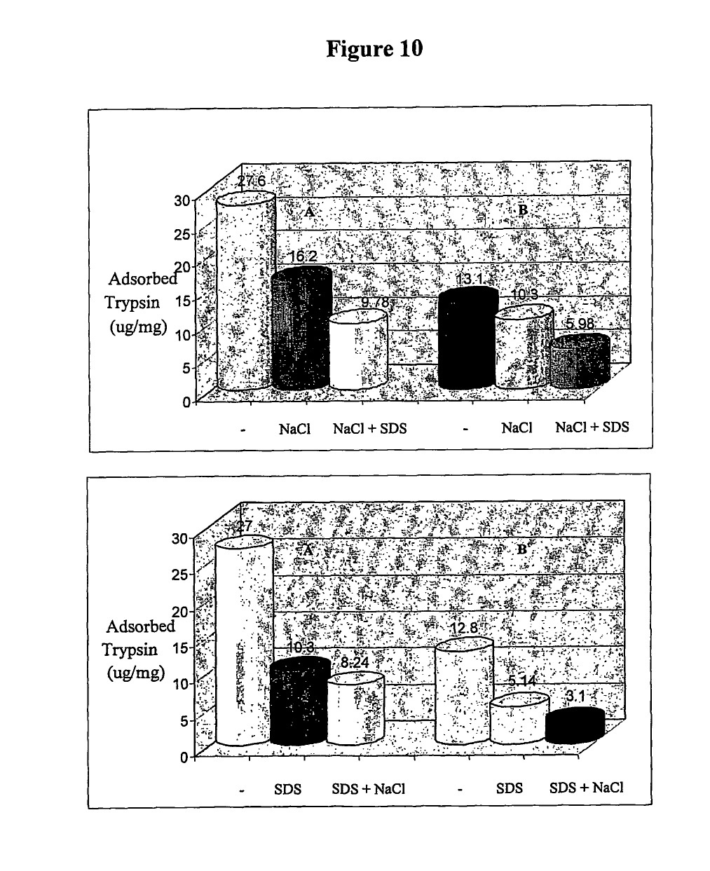

Trypsin adsorption on acid microparticle surface is a reversible interaction: protein can be easily recovered in high amounts after complex incubation in the presence of salts and/or detergents (FIG. 10).

3. In Vitro Experiments

Tat Polypeptide

The biologically active Tat protein of HIV-1 (HTLVIII-BH10) was produced in Escherichia coli, purified as a good laboratory practice (GLP) manufactured product and tested for activity as previously described (Ensoli B, Buonaguro L, Barillari G, et al, Release, uptake, and effects of extracellular human immunodeficiency virus type 1 Tat protein on cell growth and viral transactivation, J. Virol., 1993; 67:277-87; Ensoli B, Gendelman R, Markham P, et al, Synergy between basic fibroblast growth factor and HIV-1 Tat protein in induction of Kaposi's sarcoma, Nature, 1994; 371:674-80; Fanales-Belasio E, Moretti S, Nappi F, et al., Native HIV-1 Tat protein targets monocyte-derived dendritic cells and enhances their maturation, function, and antigen-specific T cell responses, J. Immunol., 2002; 168:197-206; and Chang H C, Samaniego F, Nair B C, Buonaguro L,. Ensoli B., HIV-1 Tat protein exits from cells via a leaderless secretory pathway and binds to extracellular matrix-associated heparan sulfate proteoglycans through its basic region, AIDS, 1997; 11:1421-31). To prevent oxidation that occurs easily. because Tat contains seven cysteines, the Tat protein was stored lyophilized at −80°, and resuspended immediately before use in degassed sterile PBS (2 mg/ml) for adsorption to the microparticles, or in degassed PBS containing 0.1% bovine serum alburnine (BSA) (Sigma, St. Louis, Mo.) for serological assays, as described (Fanales-Belasio et al supra). In addition, since Tat is photo- and thermosensitive, the handling of Tat was always performed in the dark and on ice. Endotoxin concentration of different GLP lots of Tat was always below the detection limit (<0.05 EU/mg), as tested by the Limulus Amoebocyte Lysate analysis.

Tat Peptides

15 amino acid Tat-derived peptides (C-terminal amide) were synthesized using standard methods (Table 8). To predict Tat CTL epitopes for the K^{d} allele, the HLA peptide motif search (http://bimas.dcrt.nih.gov/molbio/hla_bind/) was used.

TABLE 8
Tat-derived 15-mer peptides

| | | |
|---|---|---|
| | Amino acid | |
| Peptide | position | Amino acid sequence^{a} |

| | | |
|---|---|---|
| TC27 | 1-15 | MEPVDPRLEPWKHPG |
| TC28 | 6-20 | PRLEPWKHPGSQPKT |
| TC29 | 11-25 | WKHPGSQPKTACTNC |
| TC30 | 16-30 | SQPKTACTNCYCKKC |
| TC31 | 21-35 | ACTNCYCKKCCFHCQ |
| TC32 | 26-40 | YCKKCCFHCQVCFIT |
| TC33 | 31-45 | CFHCQVCFITKALGI |
| TC34 | 36-50 | VCFITKALGISYGRK |
| TC35 | 41-55 | KALGISYGRKKRRQR |
| TC36 | 46-60 | SYGRKKRRQRRRPPQ |
| TC37 | 51-65 | KRRQRRRPPQGSQTH |
| TC38 | 56-70 | RRPPQGSQTHQVSLS |
| TC39 | 61-75 | GSQTHQVSLSKQPTS |
| TC40 | 66-80 | QVSLSKQPTSQSRGD |
| TC41 | 71-85 | KQPTSQSRGDPTGPK |
| TC42 | 76-90 | QSRGDPTGPKLEQKKK |

^{a}Peptides were designed based on HIV-1 (BH10) Tat 102 aa long.

Adsorption of Tat to the Microparticles

Microparticles were resuspended (2 mg/ml) in degassed sterile phosphate buffered saline (PBS) and stored at 4° C. prior to use.

To prepare Tat-microparticle complexes, the appropriate volume of Tat and microparticles were incubated in the dark and on ice for 60 nin, and spun at 13,000 rpm for 10 min. The pellets (Tat-microparticle complexes) were resuspended in the appropriate volume of degassed sterile PBS and used immediately.

Flow Cytometry

Microparticles (50 μg) were incubated with increasing amount of the Tat protein (0.1, 1, 2, 5 and 10 μg) in a final volume of 50 μl for 60 min at room temperature under mild agitation. Microparticles alone or microparticle-Tat complexes were spun at 13.000 rpm for 15 min, washed twice and resuspended in 50 μl of PBS. 5 μl of microparticles-Tat complexes or microparticles alone were then incubated for 30 min at 4° C. with a FITC-labeled anti-Tat monoclonal antibody (Intracel, Issaquah, Wash.), or with a FITC-labeled anti-Tat rabbit polyclonal antibody, prepared in house (Magnani et al., unpublished results) and analyzed by flow cytometry (FacScan Becton-Dickinson Mountain View, Calif.).

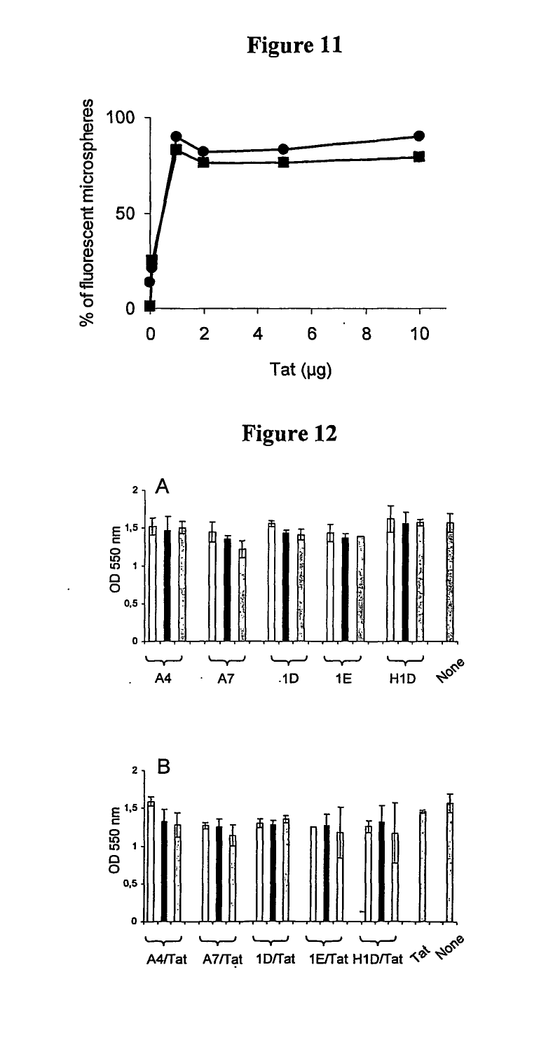

The results indicated that Tat adsorbs at the surface of the A4, A7, 1D, 1E and H1D microparticles (FIG. 11). Although the maximum fluorescence was detected with 1 μg of Tat, the result was not quantitative and did not really represent the loading efficiency of the microparticles, but it was likely due to antibody steric hindrance, as indicated by the experiments described in the following sections. Both classes of microparticles (those obtained by dispersion polymerization of styrene (monomer) in the presence of hemisuccinated poly(vinyl alcohol) and those obtained by dispersion polymerization of methyl methacrylate (monomer) in the presence of Eudragit® L100-55) were stable and could be stored as a lyophilisate or suspension for several months.

Analysis of Cytotoxicity In Vitro

Monolayer cultures of human HL3T1 cells, containing an integrated copy of plasmid HIV-1-LTR-CAT, where expression of the chloramphenicol acetyl transferase (CAT) reporter gene is driven by the HIV-1 LTR promoter, were obtained through the NIH AIDS research and reference reagents program (Bethesda, Md.) and grown in DMEM (Gibco, Grand Island, N.Y.) containing 10% FBS (Gibco).

HL3T1 cells (1×10⁴/100 μl) were seeded in 96-well plates and cultured at 37° C. for 24 h. One-hundred μl of medium containing the microparticles alone (10, 30, 50, 100, 300, 500 and 1000 μg/ml) or bound to Tat (1 μg/ml) (sextupled wells) were then added to the cells. Untreated cells and cells incubated with Tat alone were the controls. Cells were incubated for 96 h at 37°, and cell proliferation was measured using the colorimetric cell proliferation kit I (MTT based) provided by Roche (Roche, Milan, Italy) (Mosmann T., J. Immunol. Meth., 1983;65:55-63). Absorbances were measured by reading the plates at 570 nm with reference wavelength at 630 nm (OD 570/630). t-student tests were performed. Experiments were run in triplicate (SD≦10%).

Both classes of microparticles (those obtained by dispersion polymerization of styrene (monomer) in the presence of hemisuccinated poly(vinyl alcohol) and those obtained by dispersion polymerization of methyl methacrylate (monomer) in the presence of Eudragit® L100-55) and microparticle-Tat complexes were not toxic to the cells up to 50 μg/ml as compared to untreated or Tat-treated cells (p<0.01) (FIG. 12). A 50% reduction of cell viability was observed only at higher doses (300-1000 μg/ml) (data not shown).

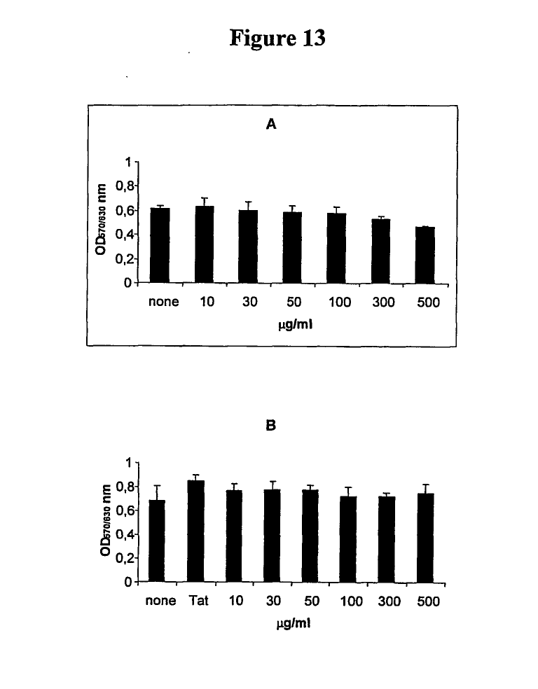

The cytotoxicity of 2H1B (acid microparticles) was also assayed in HL3T1 cells following incubation with increasing amounts of microparticles (10-500 μg/ml) as compared to untreated cells as described above. No significant reduction of cell viability was observed after 96 hours incubation in the samples treated with 2H1B, as compared to untreated cells (FIG. 13). These results indicate that 2H1B microparticles are not toxic for the cells.

Cellular Uptake of Microparticles

Isolation of murine and human primary cells was carried out as follows. 1) Six-weeks old Swiss female mice (Nossan, Italy) were injected intraperitoneally (i.p.) with 1.0 ml of 10% thioglycolate (Sigma). At 4 days, mice were sacrificed, and peritoneal exudate cells highly enriched for macrophages were harvested by i.p. lavage with 10 ml of ice-cold Hank's balanced salt solution supplemented with 10 U/ml of heparin. Cells (4×10⁶ cells) were washed twice, resuspended in DMEM supplemented with 10% heat-inactivated FBS, 1% antibiotics, 2 mM glutamine, seeded onto 35 mm Petri dishes, and incubated for 12 h in a humidified 5% CO₂ atmosphere at 37° C. to allow macrophage adherence. Nonadherent cells were gently removed with warmed DMEM medium. Monolayers were 95% pure macrophages as determined by immunostaining and surface marker analysis using a rat monoclonal antibody to mouse F4/80 (Caltag Lab., Burlingame, Calif.). 2) Murine splenocytes were purified from spleens of 10-weeks old Balb/c female mice using Ficoll gradients (Caselli E et al., J. Immunol., 1999;162:5631-8) and grown in RPMI 1640 supplemented with 10% FBS. Human monocytes and monocyte-derived dendritic cells were purified from a buffy coat, characterized and cultured as described (Micheletti F et al., Immunol., 2002;106:395-403).

HL3T1 cells (1×10⁵) were seeded in 24-well plates containing a 12-mm glass coverslip, and incubated with fluoresceinated-H1D microparticles. After incubation, cells were washed, fixed with 4% cold paraformaldehyde and observed at a confocal laser scanning microscope LSM410 (Zeiss, Oberkochen, Germany). Image acquisition, recording and filtering were carried out using a Indy 4400 graphic workstation (Silicon Graphics, Mountain View, Calif.) as previously described (Neri L M et al., Microsc. Res. Tech., 1997;36: 179-87).

Human monocytes and monocyte-derived dendritic cells (1×10⁵), and murine splenocytes (4×10⁶) were incubated in 24-well plates with fluorescent-H1D microparticles for 24 h. After incubation, cells were washed and layered onto glass slides previously coated with poly-L-lysin (Sigma) according to manufacturer's instructions. Cells were fixed with 4% cold paraformaldehyde, stained with DAPI (Sigma) and observed with a confocal microscope, as described above, and at a fluorescent microscope Axiophot 100 (Zeiss). The green fluorescence (microparticles) was observed with a 450-490 λ, flow through 510 λ and long pass 520 λ filter; the blue fluorescence (DAPI) was observed with a band pass 365 λ, flow through 395 λ and long pass 397 λ filter. For the same microscopic field, green, blue and phase contrast images were taken with a Cool-Snapp CCD camera (RS-Photometrics, Fairfax, Va.). The three images were then overlapped using the Adobe Photoshop 5.5 program.

Murine macrophages (3×10⁶) were incubated in the presence of microparticles, at a ratio of 4 microparticles per macrophage, for 1, 2 and 4 h. Cells were extensively washed to remove non-phagocytosed microparticles, fixed with 2% parafornaldehyde and 2.5% glutaraldehyde for 30 min at 4° C., and stained with toluidine blue. Cells were observed at a phase contrast microscope (100×) to count the number of macrophages with phagocytosed microparticles.

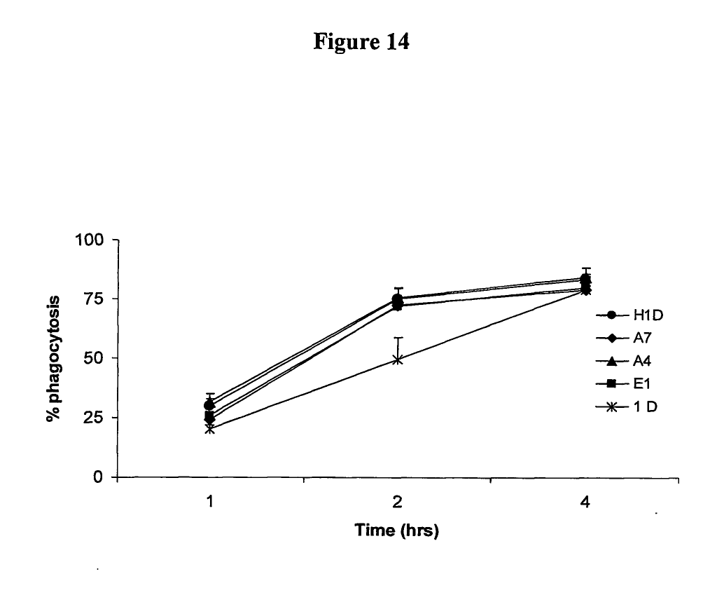

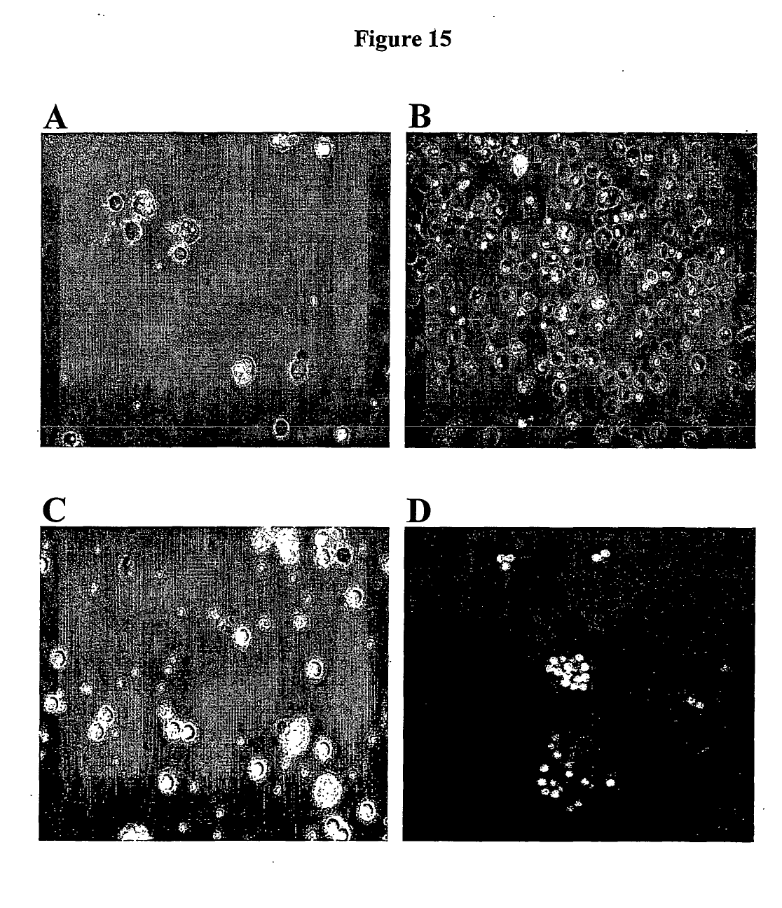

All particles were taken up by murine macrophages with similar kinetics and percentage of phagocytosis (FIG. 14). Similar results were obtained when human monocytes, monocyte-derived dendritic cells, murine splenocytes and HL3T1 cells, were cultured with fluorescent-H1D microparticles and observed with confocal and fluorescent microscopy (FIG. 15). This data indicated that the microparticles are taken up by different cell types and that chemical composition and size do not affect their phagocytosis.

Immunofluorescence

HL3T1 cells (1×10⁵) were seeded in 24-well plates containing a 12-mm glass coverstip, and incubated with fluoresceinated-H1D microparticles-Tat protein complexes. The dose of 30 μg/ml of miscrospheres associated with 5 μg/ml of Tat was used. Controls were represented by cells incubated with the Tat (5 μg/ml) protein alone or untreated cells. After incubation, cells were washed, fixed with 4% cold paraformaldehyde and analyzed by immunofluorescence with an anti-Tat monoclonal antibody (4B4C4) and a goat Cy3-conjugated anti-mouse IgG secondary serum, as previously described (Betti M et al., Vaccine, 2001;19:3408-19). Cells were colored with DAPI and observed at a fluorescence microscope. The red fluorescence (Tat) was observed with a band pass 546 λ, flow through 580 λ and long pass 590 λ filter; the green (microparticles) and blue fluorescence (DAPI) were observed as described above. For the same microscopic field, green, red, blue and phase contrast images were taken and overlapped as described above.

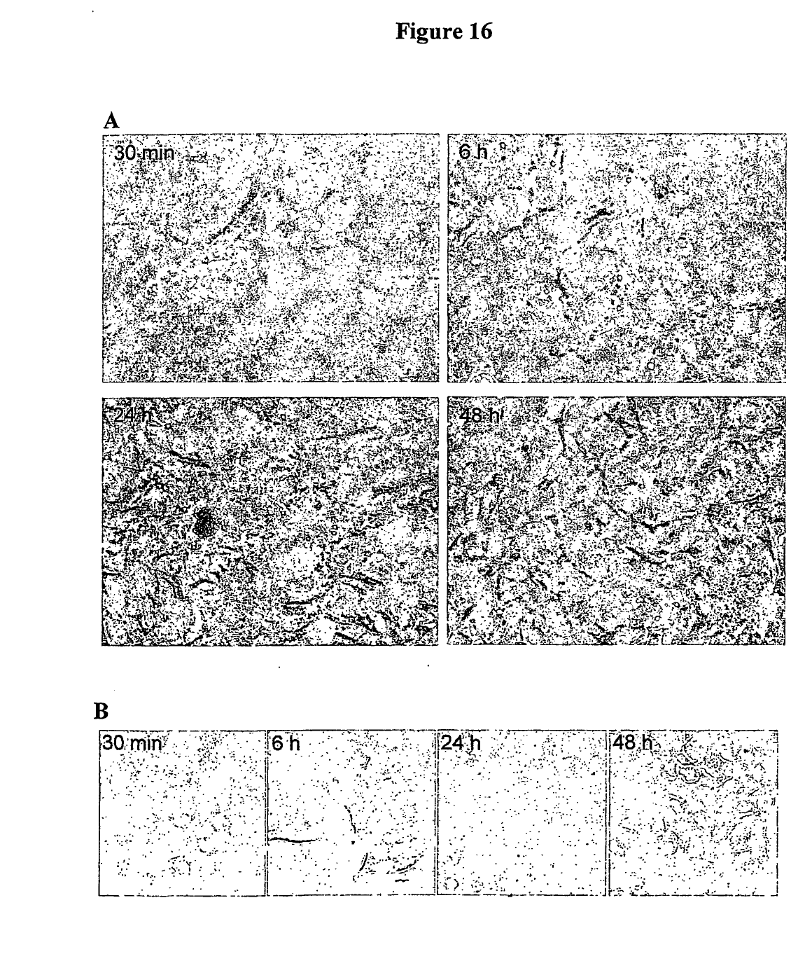

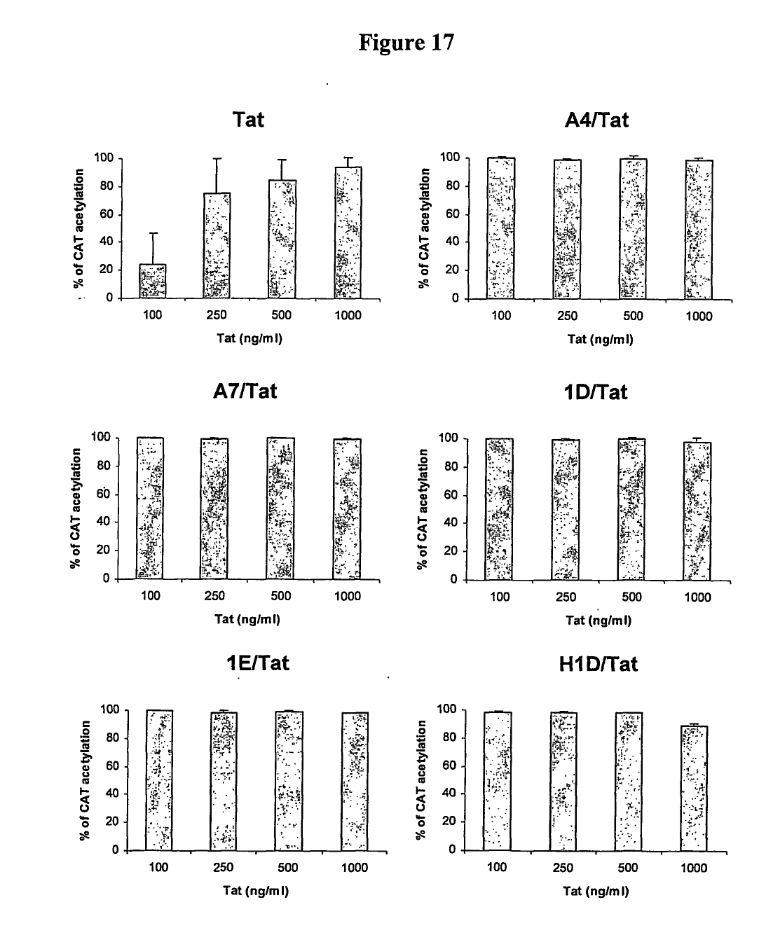

The Tat-microparticle complexes were readily taken up by the cells and the Tat protein was released intracellularly in the proximity of the nucleus (FIG. 16). Tat was released in a controlled fashion, as suggested by the observation that after 48 h Tat-loaded particles were still detectable in the cells (FIG. 17).

Evaluation of the Tat Protein Activity

HL3T1 cells (5×10⁵) were seeded in 60-mm Petri dishes. 24 h later cells were replaced with 1 ml of fresh medium and incubated with Tat alone (0.1, 0.25, 0.5, 1 μg/ml) or Tat bound to the microparticles (30 μg/ml) in the absence or presence of 100 μM chloroquine (Sigma). In some experiments, Tat alone or Tat-microparticle complexes were exposed to air and light at room temperature for 16 h before the addition to the cells. CAT activity was measured 48 h later in cell extracts after normalization to total protein content, as described previously (Betti M et al., Vaccine, 2001;19:3408-19).

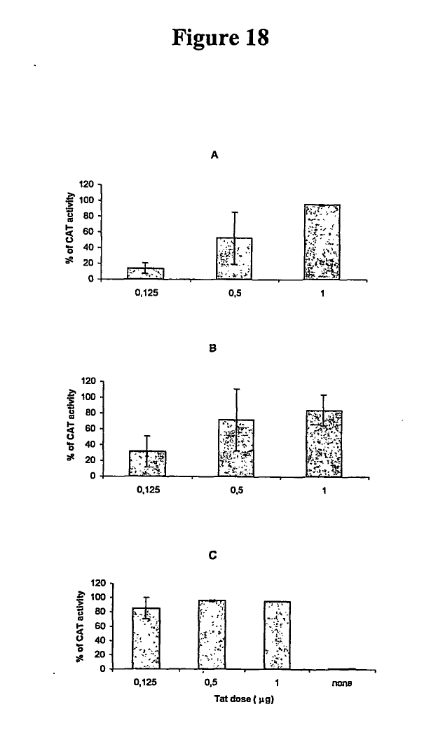

Expression of CAT was maximal and similar among all Tat-microparticle complexes (FIG. 18). In addition, at the doses of 100, 250 and 500 ng/ml of Tat bound to the microparticles, CAT expression was significantly higher than that elicited by the same doses of Tat alone (FIG. 18), suggesting that Tat bound at the surface of the microparticles is protected from proteolytic degradation and/or released in a controlled fashion from the complexes.

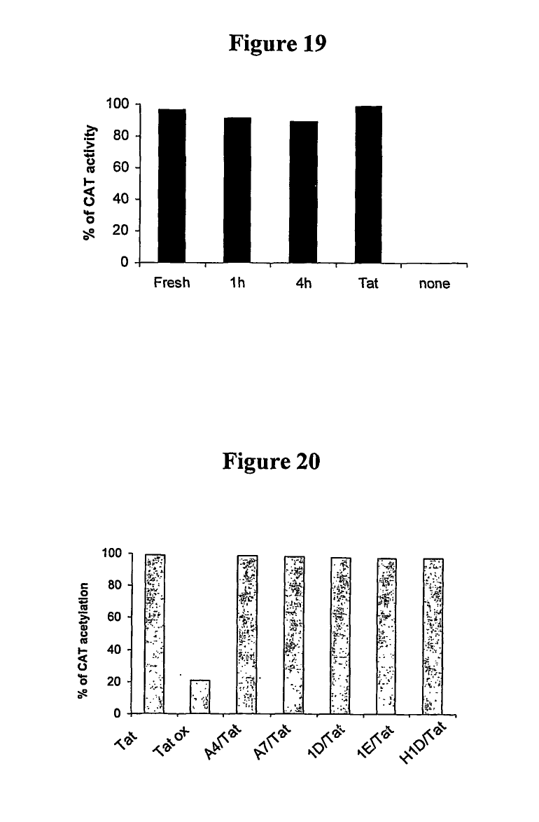

Expression of CAT was also high and similar between samples incubated with 2H1B/Tat and H1D/Tat (FIG. 19).

These results demonstrate that all the microparticles tested adsorb and release biologically active Tat protein in a dose dependent fashion, and that Tat bound to the microparticles maintains its native conformation and biological activity.

Finally, exposure to air and light did not inactivate Tat trans-activating function when Tat was previously adsorbed onto the microparticles, whereas it caused the loss of Tat biological activity when Tat was free (FIG. 20). Thus, Tat bound to the microparticles was protected from oxidation.

Evaluation of Stability of Lyophilized Microparticle/Tat Complexes

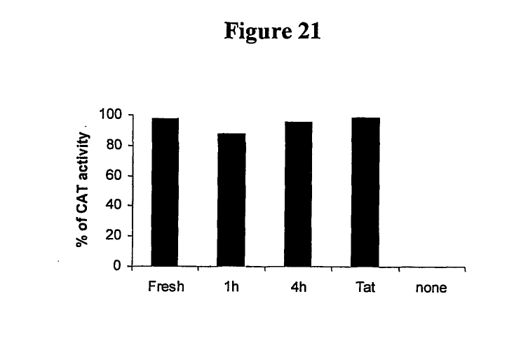

To determine whether Tat/microparticle complexes are stable in a powder form after storage at room temperature, Tat/H1D and Tat/H1D-fluo formulations were prepared, lyophilized, stored at room temperature (20-25° C.) for 15 days, resuspended in PBS and tested for Tat activity, as described in detail above (see paragraphs Analysis of cytotoxicity in vitro and Evaluation of Tat protein activity). Controls were represented by cells treated with the same formulation prepared and immediately added to the cells (fresh), or with Tat alone. The Tat/H1D and Tat/H1D-fluo complexes were stable in powder form after storage at room temperature, preserving the biological activity of the Tat protein antigen (FIGS. 20 and 21).

Gel Electrophoresis

Microparticles (50 μg) were incubated with increasing amounts of the Tat protein in a final volume of 50 μl for 60 min at room temperature under mild agitation. Microparticle-Tat complexes were spun at 13.000 rpm for 15 min, washed twice in PBS, and resuspended in 30 μl of NaCl 0.9%, phosphate buffer 5 mM. Samples were boiled for 5 min and spun at 13.000 for 15 min. Supernatants were run onto 14% SDS-polyacrylamide gels and colored with Coomassie blue (Davis L G, Dibner M D, Battey J F. In: Davis L G, Dibner M D, Battey J F, editors. Basic Methods in Molecular Biology. New York: Elsevier, 1986.).

Exposure of free Tat to oxidizing conditions caused the disappearance of the monomeric bioactive form of Tat and, concomnitantly, the appearance of oxidized Tat multimers, as compared to free Tat not exposed to air and light (data not shown). In contrast, when Tat was bound to the microparticles, the monomeric conformation of Tat was the most abundant form, either before or after exposure to air and light (data not shown). This result demonstrated that adsorption to the microparticles preserves Tat native conformation and protects it from oxidation, in agreement with the functional Tat trans-activation assay, shown earlier (FIG. 16).

4. In Vivo Experiments

A Mice Inoculation with/ H1D Fluorescent-microparticles

Animal use was according to national guidelines and institutional guidelines. Seven weeks old female BDF mice were injected with 1 mg of H1D-fluorescent microparticles resuspended in 100 μl of PBS in the quadriceps muscle of the left posterior leg. Mice were injected with 100 μl of PBS alone as control in the quadriceps muscle of the right poster leg. Fifteen and 30 minutes after injection mice were anesthetized intraperitoneally with 100 μl of isotonic solution containing 1 mg of Inoketan (Virbac, Milan, Italy), and 200 μg Rompun (Bayer, Milan, Italy), and sacrificed.

Muscles samples at the site of injections were removed, immediately submerged in liquid nitrogen for 1 minute and stored at −80° C. Five pim frozen sections were prepared, fixed with fresh 4% paraformaldehyde for 10 minutes at room temperature, washed with PBS, and colored with DAPI (0.5 μg/ml; Sigma) for 10 minutes, which stain the nuclei. After one wash with PBS, the sections were dried with ethanol, mounted in glycerol/PBS containing 1,4-diazabicyclo[2.2.2]octane to retard fading, and observed at a fluorescence microscope (Axiophot 100, Zeiss). The green fluorescence (microparticles) was observed with a 450-490 λ, flow through 510 λ and long pass 520 λ filter; the blue fluorescence (DAPI) was observed with a band pass 365 λ, flow through 395 λ and long pass 397 λ filter. For the same microscopic field, green and blue images were taken with a Cool-Snapp CCD camera (RS-Photometrics, Fairfax, Va.). The images were then overlapped using the Adobe Photoshop 5.5 program.

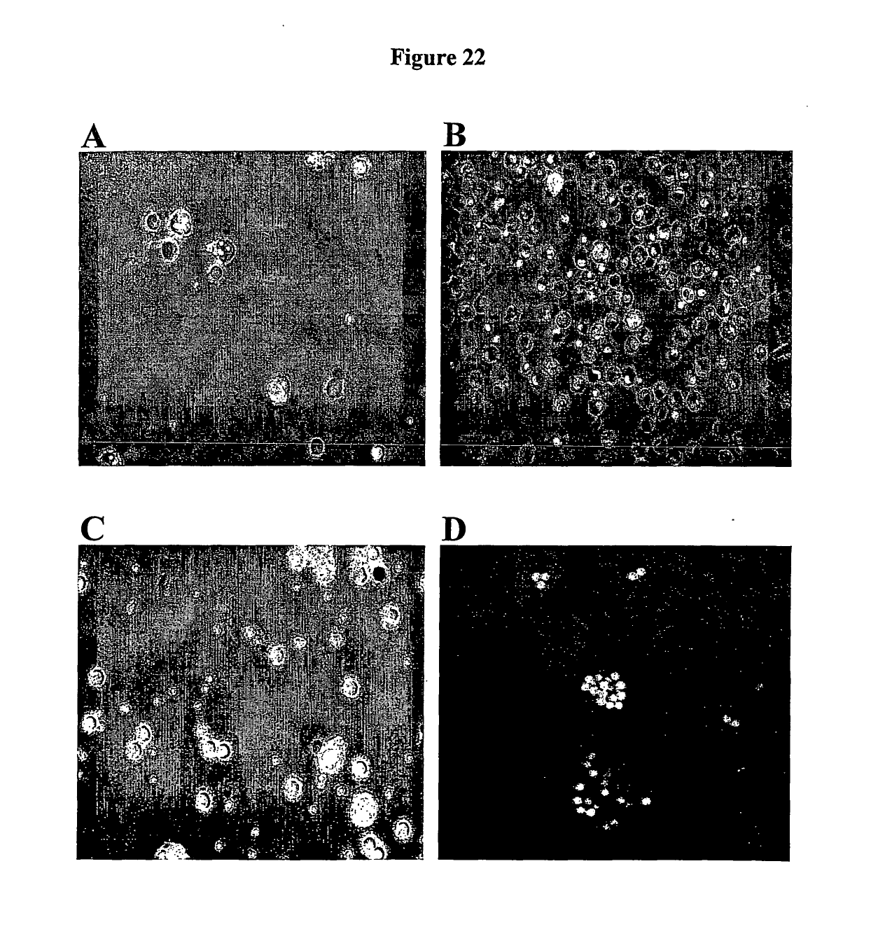

Fluorescent microparticles were readily taken up by muscle cells after injection, thus representing a useful tool for biodistribution studies (FIG. 22).

B Mice Imnmunization with Tat-adsorbed Microparticles

Animal use has complied with national guidelines and institutional policies. Seven-eight-weeks-old female Balb/c mice (H-2^{d}) Nossan, Milan, Italy) were immunized with 0.5 μg of Tat protein adsorbed to 30 μg of microparticles, Tat protein alone or Tat protein and Freund's adjuvant (CFA for the first immunization, IFA for subsequent immunizations). Control mice were injected with PBS alone. Immunogens (100 μl) were given by intramuscular (i.m.) injections in the quadriceps muscles of the posterior legs. Four separate experiments were performed. Mice were immunized at weeks 0 and 2 (2 experiments), and at weeks 0 and 4 (2 experiments). Animals were controlled twice a week at the site of injection, for the presence of edema, induration, redness, and for their general conditions, such as liveliness, vitality, weight, motility, sheen of hair. No signs of local nor systemic adverse reactions were ever observed in mice receiving the Tat-microparticle complexes as compared to mice vaccinated with Tat alone or to untreated mice. Only mice inoculated with Freund's adjuvant developed a visible granuloma at the site of injection. The immune response was evaluated two weeks after immunization. At sacrifice mice were anesthetized intraperitoneally with 100 μl of isotonic solution containing 1 mg of Inoketan (Virbac, Milan, Italy), and 200 mg Rompun (Bayer, Milan, Italy).

Anti-Tat Serology

To determine whether the chemical composition and the size of the microparticles influence the type and the strength of the immune response to HIV-1 Tat, mice (n=10) were immunized i.m. with 0.5 μg of Tat protein adsorbed to 30 μg of polystyrene (A4 and A7), and polymethyl methacrylate (iD, IE and H1D) microparticles. In addition, three groups of mice were immunized with Tat alone (n=6), Tat and Freund's adjuvant (n=10) or PBS (n=10). Two weeks after the first immunization, half number of mice by treatment group was sacrificed. At the same time, the remaining mice received the second immunization and they were sacrificed two weeks later.

Serological responses of individual mice were measured by enzyme-linked immunosorbent assay (ELISA) in 96-wells immunoplates (Nunc Immunoplate F96 Polysorp, Nunc, Naperville, Ill.). Wells were coated with 100 μl of Tat protein (1 μg/ml in 0.05 M carbonate buffer pH 9.6). Plates were sealed and incubated in the dark for 12 hours at 4° C. After extensive washes with 0.05% Tween 20 in PBS (PBS-Tween) in an automated washer (Immunowash 1575, Bio-Rad Laboratories, Hercules, Calif.), plates were blocked with 150 μl/well of PBS containing 3% BSA for 120 min at 37° C., washed and then incubated with 100 μl/well of the mice sera in duplicate wells, diluted from 1:195 up to 1:100.000, for 90 min at 37° C., and washed extensively. Immunocomplexes were detected with 100 μl/well of a horse-radish peroxidase (HRP) conjugated sheep anti-mouse IgG (Amersham Life Science, Little Chalfont, Buckinghamshire, England), diluted 1:1000 in PBS-Tween containing 1% BSA. Plates were incubated for 90 min at room temperature, washed 5 times and incubated with 100 μl/well of peroxidase substrate (ABTS) (Roche, Milan, Italy) for 40 min at room temperature. The reaction was blocked with 100 μl of 0.1 M citric acid and the absorbance was measured at 405 nm in an automated plate reader (ELX-800, Bio-Tek Instruments, Winooski, Utah). The cutoff corresponded to the mean OD₄₀₅ (+3 SD) of sera of control mice inoculated with PBS, tested in three independent assays. For anti-Tat IgG epitope mapping, eight synthetic peptides (aa 1-20, 2140, 36-50, 46-60, 56-70, 52-72, 65-80, 73-86) representing different regions of Tat (HTLVIII-BH10) were diluted in 0.1 M carbonate buffer (pH 9.6) at 10 μg/ml, and 96-well immunoplates were coated with 100 μl/well. The assays were performed as described above. The cutoff for each peptide corresponded to the mean OD₄₀₅ (+3 SD) of sera of control mice injected with PBS, tested in three independent assays.

For anti-Tat IgG isotyping, plates were coated with Tat protein and incubated with mice sera diluted 1:100 and 1:200, as described above. After washing, 100 μl of goat anti-mouse IgG1, or IgG2a (Sigma), diluted 1:100 in PBS-Tween containing 1% BSA, were added to each well. Immunocomplexes were detected with a horse-radish peroxidase-labeled rabbit anti-goat IgG (Sigma) diluted 1:7500 in PBS-Tween containing 1% BSA, as described above. The cutoff for each IgG subclass corresponded to the mean OD₄₀₅ (+3 SD) of sera of control mice injected with PBS, tested in three independent assays.

Serum antibody responses were monitored by ELISA at sacrifice. All five groups of mice immunized with the Tat/microparticle complexes developed specific anti-Tat antibodies, that were detectable after the second imrnunization and with titers similar among the five treatment groups and to Tat-vaccinated mice (Table 9).

TABLE 9
Humoral immune response to Tat protein after immunization
with Tat/microparticle complexes^{a}

| Group | I Immunization | II Immunization |
|---|---|---|
| A4/Tat | 0/5 | 5/5 |
| | (0) | (2109 ± 2611) |
| A7/Tat | 0/5 | 3/5 |
| | (0) | (624 ± 652) |
| 1D/Tat | 0/5 | 5/5 |
| | (0) | (4687 ± 2210) |
| 1E/Tat | 0/5 | 5/5 |
| | (0) | (1093 ± 1270) |
| H1D/Tat | 0/5 | 5/5 |
| | (0) | (6874 ± 10.385) |
| Tat | 2/3 | 3/3 |
| | (130 ± 112) | (9635 ± 13.358) |
| ^{a}Mice were immunized once (I immunization) or twice (II immunization), at weeks 0 and 2, and sacrificed two weeks later. The antibody response was determined on serially diluted sera of individual mice by ELISA using Tat protein as the antigen. Results of one representative experiment are expressed as | | |
| # the number of responder mice vs the total number of immunized mice. In each group the mean titers ± SD of the responders are reported in parenthesis. The differences in Ab titers of mice immunized with the Tat/microparticle complexes as compared to mice vaccinated with Tat alone were not significant (p > 0.01). | | |

The epitope reactivity of the antibodies was directed to the NH₂-terminal region of the protein (residues 1-20) in all mice of all treatment groups immunized with the Tat/microparticle complexes, or Tat. A second reactive epitope was identified at residues 21-40 only in the serum of two mice, one immunized with A4/Tat (mouse ID 10) and the other immunized with ID/Tat (mouse ID 9) (data not shown).

The isotype analysis of the IgG subclasses indicated the presence of both IgG1 and IgG2a isotypes. However, a prevalence of the IgG1 subclass was observed in all groups (data not shown).

Tat-specific T Cell Activation

Mononuclear cells were purified from spleens using cells strainers provided by Falcon. Cells were resuspended in PBS containing 20 mM ED TA, treated with a red blood cells lysis buffer (100 mM NH₄Cl, 10 mM KH CO₃, 10 mM EDTA) for 4 minutes at room temperature, and washed twice with RPMI 1640 (Gibco) without serum. Cells were resuspended in RPMI 1640 supplemented with 10% heat-inactivated FBS (Hyclone), and counted by trypan blue exclusion dye. Purified splenocytes were pooled by treatment group, and used to evaluate the cellular immune responses.

Tat-specific T-cell activation was determined using different assays.

1) Splenocytes were cultured at 2×10⁵/well (sextupled wells) in 200 μl of RPMI 1640 supplemented with 10% heat inactivated FBS in the presence of Tat protein (0. 1, 1 or 5 μg/ml) or Con A (10 μg/ml) (Sigma) for five days. Methyl-³H-thymidine (2.0 Ci/mmol; ICN) was added to each well (1 μCi) and cells were incubated for 16 h. [³H]-Thymidine incorporation was measured with a β-counter. The S.I. was calculated by dividing the mean cpm of six wells of antigen-stimulated cells by the mean cpm of six wells of the same cells grown in the absence of the antigen. Values higher than the cutoff (mean S.I. (+2 SD) of the control mice injected with PBS alone] were considered positive.

2) Stable clones of murine Balb/c 3T3-Tat expressing cells and Balb/c 3T3-pRPneo-c (referred to as BALB/c-control cells) (H^{2d} haplotype) were grown in Dulbecco's minimal essential medium plus 10% FBS and G418 (350 μg/ml, Sigma). Mice splenocytes were co-cultivated at 20:1 ratio with BALB/c 3T3-Tat expressing cells in the presence of Tat (0.5 μg/ml). After 4 days of culture, rIL-2 (10 U/ml; Roche, Milan, Italy) was added to the cultures and cells grown for additional 48 hrs. γINF production was measured by ELISA on culture supernatants before and after addition of IL-2. Ninety-six wells immunoplates Nunc lmmunoplate F96 Polysorp) were coated with 100 μl of an anti-mouse γINF mAb (1 μg/ml; Endogen, Woburn, Mass.) in 0.03 M carbonate buffer for 16 h at 4° C. Empty wells were then blocked with 200 μl of PBS-4% BSA (assay buffer) for 1 h at room temperature, extensively washed with PBS-0.05% Tween 20 (washing buffer), and incubated with 50 μl of serially diluted cell supernatants for 1 h at room temperature. A titration curve (from 0 up to 20.000 μg/ml of recombinant murine γINF-gamma, Euroclone, Devon, U.K.) was included in each plate. Each sample was tested in duplicate. Empty plates were then incubated with 50 μl/well of a biotine-labelled anti-mouse γINF mnAb (400 ng/ml in assay buffer; Endogen) for 1 h at room temperature, extensively washed and incubated with HRP-labelled streptavidin (Endogen) diluted 1:6000 in assay buffer for 30 min at room temperature. Plates were washed, incubated with 100 μl/well of 3,3′,5,5′-tetramethyl-benzidine (TMP; Sigma) substrate for 3 min, blocked with 100 μl/well 3 N HCl and the adsorbency read at 450 nm.

3) To measure the T-cell proliferation in response to Tat-derived 15-mer peptides, containing the computer predicted CTL epitopes for K^{d} allele, irradiated spleen cells (5×10⁵) from naive syngeneic Balb/c mice (serving as APC) were incubated in 96-flat bottom wells with 2×10⁵ M of each Tat peptide for 1 hour. Splenocytes (1×10⁵) from immunized mice, previously co-cultivated for 4 days with BALB/c 3T3-Tat expressing cells (at 20:1 ratio) in the presence of Tat (0.5 μg/ml) and purified using Ficoll gradients, were added to the wells in a final volume of 200 μl and final peptide concentration of 10⁻⁵ M. After 24 hours, aliquots of culture media were collected to measure the release of γIFN, whereas after additional 72 hours of culture cells were pulsed with methyl-³H-thymidine (1 μCi/well) for 24 hours. Incorporated radioactivity was measured by liquid scintillation spectroscopy.

Thus, CD4+T-cell proliferation in response to Tat was evaluated using mice splenocytes. Splenocytes of mice, obtained two weeks after the first or the second immunization, were cultured five days with 0. 1, 1 and 5 mg/ml of Tat protein. Antigen-stimulated T-cell proliferation was determined by [³H]thymidine incorporation (Table 10). After one immunization, specific responses to the highest dose of Tat were observed in splenocytes of all groups immunized with the Tat/microparticle complexes, and Tat. In addition, for the A7/ and 1E/Tat treatment groups Tat-specific CD4+T-cell responses were detected also at the lower dose of 1 μg/ml of Tat. After two immunizations, Tat-specific T-cell proliferation was detected at both 1 and 5 μg/ml of Tat in all groups with and without the microparticles, and in addition, mice immunized with A4/Tat and 1D/Tat responded to as little as 0.1 μg/ml of recombinant Tat.

TABLE 10
Lymphoproliferative response to Tat protein after
immunization with Tat/microparticle complexes^{a}

| | I Immunization | | | | II Immunization | | | |
|---|---|---|---|---|---|---|---|---|
| | Tat 0.1 | Tat 1 | Tat 5 | ConA 2 | Tat 0.1 | Tat 1 | Tat 5 | ConA 2 |
| Group | μg/ml | μg/ml | μg/ml | μg/ml | μg/ml | μg/ml | μg/ml | μg/ml |
| A4/Tat 0.5 | 1.08 | 2.66 | 12.71 | 21.14 | 2.02 | 13.02^{b} | 15.62 | 19.52 |
| A7/Tat 0.5 | 1.51 | 3.21 | 19.05^{b} | 33.43 | 0.7 | 1.79 | 9.24 | 27.21 |
| 1D/Tat 0.5 | 0.81 | 1.73 | 14.49 | 67.38 | 6.60 | 15.66^{b} | 25.71 | 31.71 |
| 1E/Tat 0.5 | 1.30 | 4.17 | 13.37^{b} | 15.34 | 1.64 | 4.9 | 11.59 | 16.65 |
| H1D/Tat 0.5 | 1.55 | 2.44 | 31.95^{b} | 38.97 | 1.7 | 3.51 | 14.25 | 20.11 |
| Tat | n.d. | 2.86 | 6.2^{b} | 75.8 | n.d. | 4.3^{b} | 27.03 | 40.6 |
| PBS | 1.67 | 2.66 | 4.04 | 5.3 | 1.84 | 1.46 | 5.6 | 18 |

^{a}Mice were immunized at weeks 0 and 2, and immune response tested two weeks after the first and the second immunization. Cells were stimulated with recombinant Tat protein or ConA. Values represent the SI of murine splenocytes (pool of 5 spleens) after Tat or ConA activation. A SI higher than that of the control group injected with PBS was considered positive.
^{b}The differences in proliferative responses vs mice immunized with Tat alone were significant (p < 0.05).

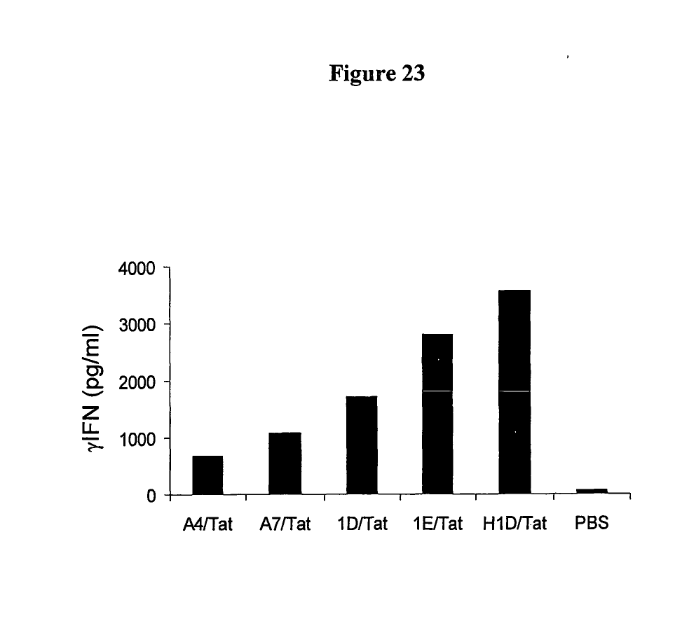

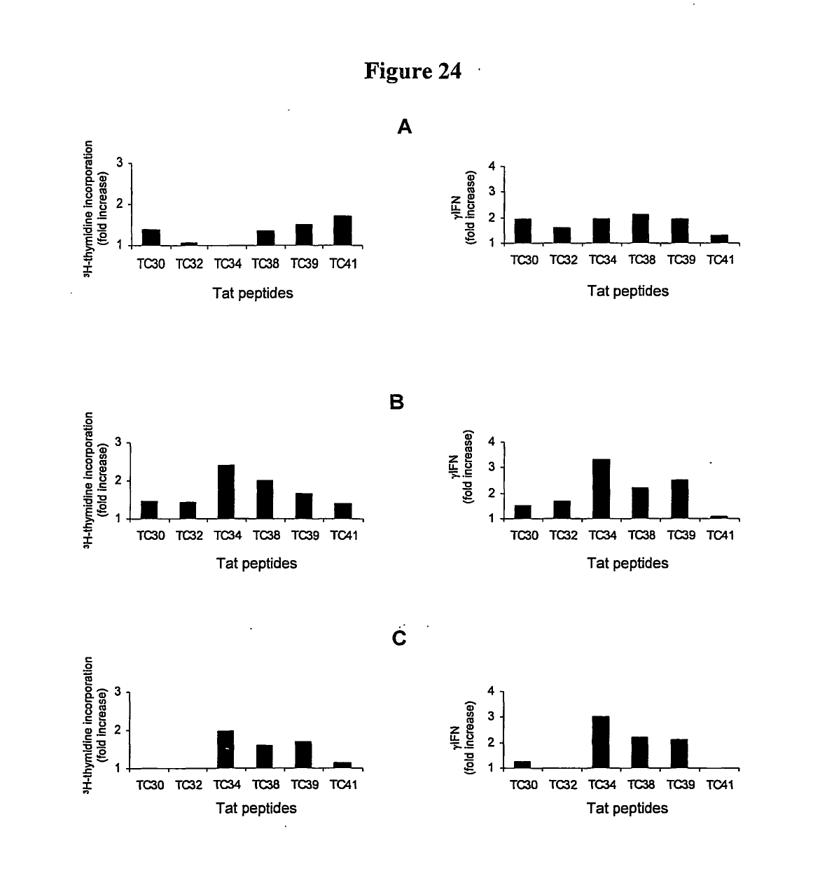

In separate experiments, mice were immunized twice (at week 0 and 4) with the Tat/microparticle complexes. Splenocytes of mice, obtained two weeks after the second immunization, were co-cultured with BALB/c 3T3-Tat expressing cells in the presence of Tat. After 4 days of culture, the production of γINF in culture media of restimulated spleen cells was measured by ELISA. As shown in FIG. 23, γINF production resulted significantly increased in all five groups immunized with the Tat/microparticle complexes, as compared to mice injected with PBS. This effect comparable between the PS particles (A4 and A7) and, among the PMMA particles, it was greatly evident in the H1D/Tat treatment group. Thus, we measured the T-cell proliferation in response to Tat-derived peptides in two treatment groups, one for each type of microparticles. Splenocytes of mice vaccinate A4/Tat and H1D/Tat vaccinated mice, after co-cultivation with BALB/c 3T3-Tat expressing cells in the presence of Tat for 4 days, were purified and co-cultured with irradiated naive splenocytes pulsed with several Tat peptides. T cell proliferation was measured by ³[H]thymidine incorporation after 96 hrs of culture, and γINF release was tested on aliquots of culture supernatants collected after 24 hrs of culture. The results of these experiments showed specific cell proliferation and release of γINF in response to TC34, TC38 and TC39 Tat peptides, containing computer predicted CTL epitopes for the K^{d} allele, in a fashion similar to Tat treated mice (FIG. 24). In addition, although weaker, responses to other Tat peptides, including TC30, TC32, and TC41, were observed (FIG. 24). Responses to other Tat-peptides were not observed (not shown).

Evaluation of the Safety of Tat-microparticle Complexes In Vivo

At sacrifice animals were subjected to autopsy. Samples of cutis, subcutis and skeletal muscles at the sites of injection and other organs (lungs, heart, intestine, kidneys, spleen and liver) were fixed in 10% formalin for 12-24 h, embedded in paraffin, and routinely processed for histological examination. Three-5 μm paraffin-embedded sections were stained with hematoxylin and eosin, subjected to periodic acid-Shiff (PAS) reaction with and without diastase treatment (Sigma). Serial tissue sections were immuno-stained using the avidin-biotin-peroxidase complex technique (Vectastain ABC Kit PK-4002, Vector Labs, Burlingame, Calif.) according to Hsu et al. (J. Histochem. Cytochem. 1981;29:577-80). The panel of antibodies included S-100 (Dako, Denmark), HH-F 35 (Dako) for detection of α-actin, CD68 and Mac387 (Dako) for detection of macrophages. Briefly, after deparaffinization and rehydration, endogenous peroxidase was blocked with 0.3% H₂O₂ in methanol; samples were then incubated with primary antibodies for 10-12 h at 4° C. Biotinilated-anti-mouse and anti-rabbit immunoglobulins (Sigma) were utilized as secondary antibodies. Specific reactions were detected following incubation with avidin-biotin-peroxidase conjugated and treatment with diaminobenzidine (Sigma) and hydrogen peroxide.

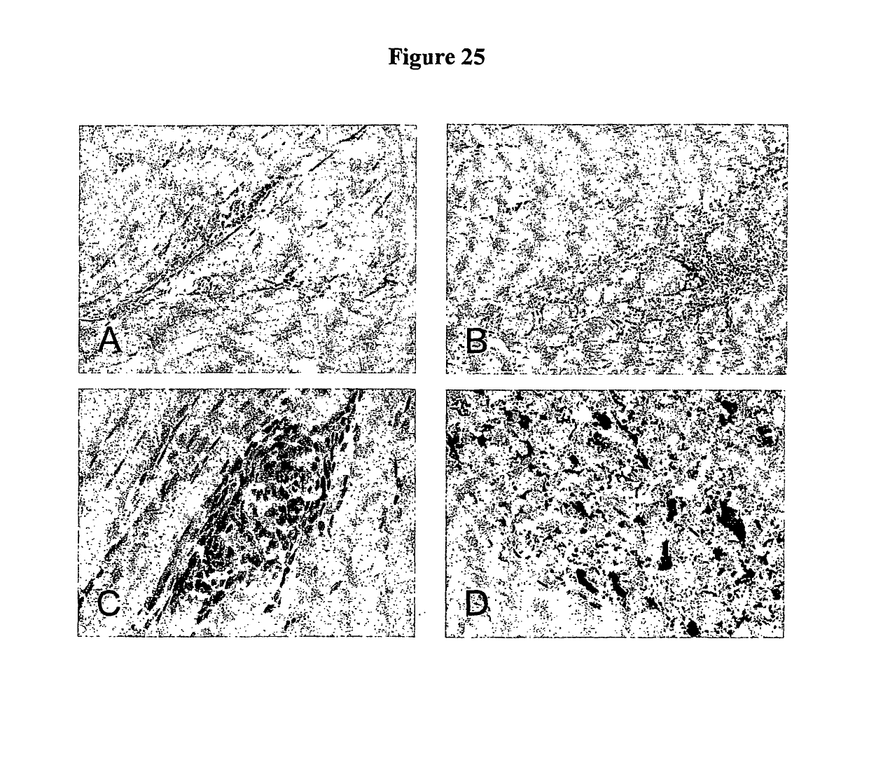

Histologically two types of pictures were observed at the site of injection. The first consisted of small foci, involving one or two muscle fibers, showing increased number of nuclei, and scarce macrophage infiltrate in the interstitial space (FIG. 25A and C). These features were prevalently detected in mice injected with the Tat-microparticle complexes or Tat alone. The second type of picture was found in the muscular fascia and in the surrounding adipose tissue, and it was characterized by a central area of necrosis surrounded by neutrophil granulocytes and macrophages (FIG. 25B and D). The macrophages always showed good reactivity to CD68 and Mac387 monoclonal antibodies; T and B lymphocytes were not detected in the inflammatory reactions. This type of lesion, as well as the higher number of inflammatory cells, was detected in the majority of mice receiving Tat and Freund's adjuvant. In the other animals and in control mice inoculated with PBS, the inflammatory reaction was inconspicuous, related to the traumatic stimulus or absent (data not shown). Laden macrophages reaction or other type of inflammatory reactions were not observed in the other organs.

No differences in the inflammatory reactions, related to the chemical composition and size of microparticles or the dose of Tat, were detected after one immunization. Indeed, only 2/22 (9%) mice, inoculated with A4-Tat 0.5 μg or 1D-Tat 0.5 μg, showed an inflammatory reaction. After two immunizations, 14/47 (30%) mice treated with the microparticle-Tat complexes developed a local inflammatory reaction. After three immunizations, 23/38 (60%) of mice treated with the Tat-microparticle complexes showed variable inflammatory reactions at the site of inoculation. In conclusion, the frequency of the inflammatory reactions correlated with the number of immunizations.

Tat-treated mice presented local inflammation (type one picture) only after the. second inoculation in about 50% of the mice; macrophages infiltration was more frequently observed, but it was not related to the dose of Tat.

All mice treated with Tat and Freund's adjuvant showed intense inflammatory reactions independently from the number of immunizations; the incidence was more than 70% after the first injection and raised up to 90-100% after the second and the third treatment. This is likely due to the type of adjuvant used.

C Mice Immunization with Ovalbumin-adsorbed Microparticles

Protein

Ovalbumin was purchased from Sigma (cat. A-2512; St. Louise, Mo.). Ovalbumin molecular weight and isolectric point are 45.000 Daltons and 4.63 (Merck Index), respectively. The protein was resuspended (2 mg/ml) in phosphate buffered saline (PBS) and stored at 4° C. The protein sequence is shown in SEQ ID NO: 52.

Ovalbumin Peptides

Ovalbumin peptides (Table 11) were synthesized by UFPeptides s.r.l. (Ferrara, Italy). Stocks were prepared in DMSO at 10⁻² M concentration, kept at −80° C., and diluted in PBS immediately before use.

TABLE 11
Ovalbumin peptides

| Peptide | Ovalbumin | Peptide | Class I | |
|---|---|---|---|---|
| ID | (aa) | sequence | restriction | Reference |
| CFD | 11-18 | CFDVFKEL | H-2K(b) | Lipford et al. |
| | | | | J. Immunol. 1993, |
| | | | | 150: 1212-1222 |
| KVV | 55-62 | KVVRFDKL | H-2K(b) | Mo et al. J. |
| | | | | Immunology. 2000, |
| | | | | 164: 4003-4010 |
| SII | 257-264 | SIINFEKL | H-2K(b) | Catipovic et al. |
| | | | | J. Exp. Med. 1992, |
| | | | | 176: 1611-1618 |
| OVA1 | 25-32 | ENIFYCPI | H-2K(b) | Chen et al. |
| | | | | J Exp. Med. 1994, |
| | | | | 180: 1471-1483 |
| OVA2 | 107-114 | AEERYPIL | H-2K(b) | Lipford et al. |
| | | | | J. Immunol. 1993, |
| | | | | 150: 1212-1222 |
| | | | | Chen et al. |
| | | | | J Exp. Med. 1994, |
| | | | | 180: 1471-1483. |
| OVA3 | 176-183 | NAIVFKGL | H-2K(b) | Lipford et al. |
| | | | | J. Immunol. 1993, |
| | | | | 150: 1212-1222 |
| | | | | Chen et al. |
| | | | | J Exp. Med. 1994, |
| | | | | 180: 1471-1483 |

Ovalbumin/Microparticle Complex Formation

HE1D microparticles (lyophilized powder) were resuspended in sterile PBS at 2 mg/ml at least 24 hours before use. The appropriate volumes of ovalbumin and HE1D microparticles were mixed and incubated for 2 hours at room temperature. After incubation samples were spun at 13.000 rpm for 10 minutes. The pellets (ovalbumin/HE1D complexes) were resuspended in the appropriate volume of PBS and used immediately.

Gel Electrophoresis

HE1D microparticles (30 μg) were incubated with increasing amounts of ovalbumin for 2 hours at room temperature under mild agitation. HE1D/ovalbumin complexes were spun at 13.000 rpm for 15 min. Supernatants (unbound protein) were collected and analyzed by SDS-PAGE. Pellets (HE1D/ovalbumin complexes) were washed twice in PBS, and resuspended in 30 μl of NaCl 0.9%, phosphate buffer 5 mM. Samples were boiled for 5 min and spun at 13.000 for 15 min. Supernatants (bound protein) were run onto 14% SDS-polyacrylamide gels and analyzed by silver staining (Davis L G, Dibner M D, Battey J F. In: Davis L G, Dibner M D, Battey J F, editors. Basic Methods in Molecular Biology. New York: Elsevier, 1986). Quantification was carried out using a densitometer gel analyzer (Quantity-One, BioRad Laboratories, Milan, Italy) as compared to known amounts of ovalbumin migrated in each gel.

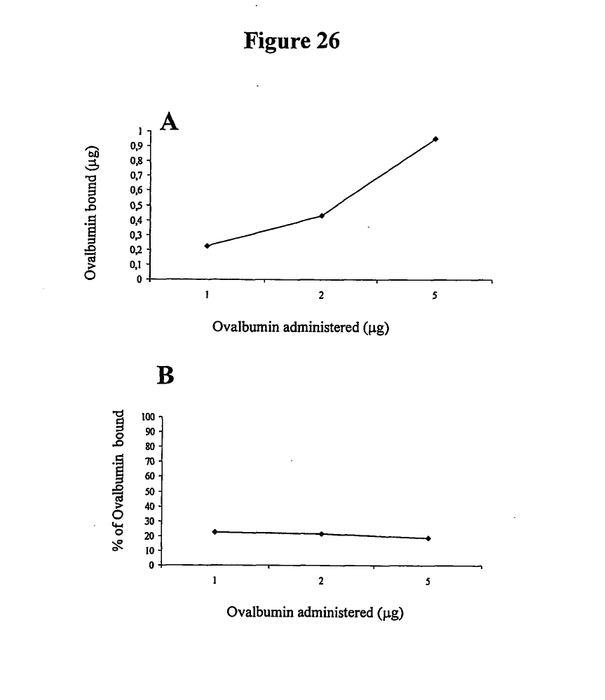

The results indicated that ovalbumin adsorbs at the surface of these basic microparticles in a dose-dependent fashion (FIG. 26A), with an adsorption efficiency of approximately 20% (FIG. 26B).

Mice Immunization

Animal use was according to national guidelines and institutional policies. Seven-weeks-old female C57BL6/J (H^{2kb}) mice (Harlan, Udine, Italy) were immunized subcutaneously in 1 site with 100 μl of immunogens, as described in Table 12. One group of mice was immunized with the Ovalbumin/HEl D complexes. Two groups of mice were immunized with Ovalbumin and Freund's or Alum adjuvants. These two groups were included to compare the immunogenicity of the complexes to that induced by commonly used adjuvants, for which Ovalbumin CTL immune responses are well characterized. In addition, to determine whether HE1D microparticles can be used to deliver peptides for vaccination purpose, the SII peptide, which contains an immunodominant ovalbumin CTL epitope, was adsorbed onto HE1D microparticles, and used to immunize mice. Finally, one group of mice was immunized with with SII and Freund's adjuvant. Controls were injected with PBS alone. Immuniogenes were given by the subcutaneous route at days 1 and 14, and sacrificed 10 days later.

TABLE 12
Immunization protocol

| | | | Schedule of |
|---|---|---|---|
| | Immunogen | | immunization |
| Immunogen | dose | Route | (days) |
| PBS + | — | subcutaneous | 1, 14 |
| Freund's | | | |
| Ovalbumin + | 1 μg | subcutaneous | 1, 14 |
| Freund's | | | |
| Ova protein + | 1 μg | subcutaneous | 1, 14 |
| Alum | | | |
| SII + | 1 μg | subcutaneous | 1, 14 |
| Freund's | | | |
| Ovalbumin/ | 1 μg/30 μg | subcutaneous | 1, 14 |
| HE1D | | | |
| SII/HE1D | 1 μg/30 μg | subcutaneous | 1, 14 |

During the course of the experiments, animals were controlled twice a week at the site of injection and for their general conditions (such as liveliness, food intake, vitality, weight, motility, sheen of hair). No signs of local nor systemic adverse reactions were ever observed in mice receiving the protein/ or the peptide/HE1D complexes as compared to mice vaccinated with ovalbumin and Freund's or alum, or to mice injected with PBS.

IFN-γ Elispot

Splenocytes were purified from spleens squeezed on filters (Cell Strier, 70 μm, Nylon, Becton Dickinson). Cells were resuspended in RPMI 1640 containing 10% FBS and used for the analysis of cytitoxic responses (CTL) by IFNγ Elispot. Pool of 3 spleens per each experimental group were used.

IFN-γ Elispot was carried out using a commercially available kit provided by Becton Dickinson (murine IFNgamma ELISPOT. Set; BD Pharmingen; Cat#551083), according to manufacturer's instructions. Briefly, nitrocellulose 96-well plates were coated with 10 μg/ml of anti-IFN-γ mAb overnight at 4° C. The following day the plates were washed 4 times with PBS, and blocked with RPMI 1640 supplemented with 10% foetal bovine serum for 2 hours at 37° C. Splenocytes (2.5 and 5×10⁵/200 μl) were purified and immediately added to the wells (triplicate wells) and incubated with ovalbumin peptides (10⁶ M) (SII, KVV, CFD, OVA1, OVA2, OVA3) for 16 hours at 37° C. Controls were represented by cells incubated with Concanavaline A (Sigma; 5 μg/ml) (positive control) or with medium alone (negative control). The spots were read using an Elispot reader (Flivis, Germany). The results are expressed as neat number of spots (SFU)/10⁶ cells [mean number of spots of peptide treated wells minus the mean number of spots of the negative control which corresponded to: Ova+Freund's 20 SFU/10⁶ cells; Ova+Alum 45 SFU/10⁶ cells; SII+Freund's 40 SFU/10⁶ cells; Ova/HE1D 150 SFU/10⁶ cells; SII/HE1D 150 SFU/10⁶ cells, respectively].

The results are shown in Table 13 below. For each peptide, the negative control was always below 10 spots/10⁶ cells. Results are expressed as the number of spots (SFU)/10⁶ cells subtracted of the SFU/10⁶ cells of the negative controls. Responses ≧30 SFU/10⁶ cells are considered positive. The results indicate that both Ovalbumin/HE1D and SII/HE1D complexes are immunogenic and elicit CTL responses which are comparable to those induced by 2 adjuvants which are known to induce good CTL responses when they are inoculated with Ovalbumin. In addition, these results indicate that microparticles can be used for peptide delivery.

TABLE 13
Results of the IFNγ Elispot

| | Immunogens | | | | |
|---|---|---|---|---|---|
| | Ova + | Ova + | Ova/ | SII + | SII/ |
| Peptide | Freund's | Alum | HE1D | Freund's | HE1D |
| SII | 15 | 29 | 9 | 46 | 45 |
| KVV | 20 | 8 | 52 | nt | nt |
| CFD | 47 | 54 | 54 | nt | nt |
| OVA1 | 30 | 32 | 0 | nt | nt |
| OVA2 | 15 | 40 | 21 | nt | nt |
| OVA3 | 35 | 7 | 91 | nt | nt |

nt, not tested.

D Monkey Immunization with Tat-adsorbed Microparticles
Tat Protein and Peptides

The 86-aa long Tat protein (HTLVIIIB, BH-10 clone) was expressed in Escherichia coli and isolated by successive rounds of high pressure chromatography and ion-exchange chromatography, as previously described. The purified Tat protein is >95% pure as tested by SDS-PAGE, and HPLC analysis. To prevent oxidation that occurs easily because Tat contains seven cysteines, the Tat protein was stored lyophilized at −80° C. and resuspended in degassed sterile PBS (2 mg/ml) immediately before use. In addition, since Tat is photo- and thermo-sensitive, the handling of Tat was always performed in the dark and on ice. Tat peptides (15-mers overlapping by 10 residues) spanning the entire Tat sequence (aa 1-102) were synthesized by UFPeptides s.r.l. (Ferrara, Italy). Peptide stocks were prepared in DMSO at 10⁻² M concentration, kept at −80° C., and diluted in PBS immediately before use.

Immunization Protocol and Schedule

Based on these results in the murine model, H1D particles were selected to undergo a pilot experiment in monkeys. Thus, safety and immunogenicity studies were carried out in cynomolgus macaques (Macaca fascicularis), a nonhuman primate model closer to human than rodents. Three groups of monkeys (n=3) were included in this study (Table 14). Group A animals were immunised 6 times (weeks 0 and 4, 12, 18, 21, 35) subcutaneously with 10 μg of Tat protein and Alum. Group B macaques were immunised intramuscularly 4 times with 10 μg of Tat protein conjugated to 60 μg of H1D microparticles (weeks 0, 4, 12, and 18) and boosted subcutaneously twice (week 21 and 35) with 10 μg of Tat protein and Alum. Group C animals represented the control, and were inoculated 4 times intramuscularly with 60 μg of H1D microparticles alone, and once subcute with Alum alone.

TABLE 14
Vaccination protocol

| | Mk | Immunizations | |
|---|---|---|---|
| Group | code | (4) | Boosters (2) |
| A | L162F | 10 μg Tat + | 10 μg Tat + |
| Tat + ALUM | T197B | 250 μL ALUM | 250 μL ALUM |
| | BA327C | (Total volume: | (Total volume: |
| | | 500 μL, s.c.) | 500 μL, s.c.) |
| B | M77OF | 10 μg Tat + | 10 μg Tat + |
| H1D-Tat | O854G | 60 μg H1D | 250 μL ALUM |
| Tat + ALUM | BD765B | (Total volume: | (Total volume: |
| | | 500 μL, i.m.) | 500 μL, s.c.) |
| | | (250 μL per site) | |
| C | AC032 | 60 μg H1D | 250 μL ALUM |
| H1D or ALUM | AC739 | (Total volume: | (Total volume: |
| | AC924 | 500 μL, i.m.) | 500 μL, s.c.) |
| | | (250 μL per site) | |

s.c. = subcute;
i.m. = intramuscular

None of the animals experienced any local or systemic adverse reaction nor showed signs of inflammation, distress or sufference, as assessed by daily clinical monitoring and monthly blood chemistry measurements, upon single or multiple inoculations of Tat protein adsorbed onto H1D microparticles (H1D-Tat) (Tables 15 and 16).

TABLE 15
Vital signs and parameters monitored (daily and every
2-4 weeks, respectively) after each injection of Tat
protein formulated with Alum or H1D microparticles
in cynomolgus monkeys (Macaca fascicularis).

| | |
|---|---|
| Diarrhea | Body weight |
| Vomiting | Complete blood cell count (CBC) |
| Pruritis/rash | Absolute number and percentage of |
| | peripheral blood lymphocyte subsets |
| | (CD3, CD4, CD8, CD20, CD56) |
| Fever | Routine biochemical parameters (glucose, |
| (T ≧ 38.5° C.) | cholesterol, blood urea nitrogen (BUN), |
| Tenderness | bilirubin total and direct, aspartate |
| Erythema | aminotransferase (AST), alanine amino- |
| Warmth | transferase (ALT), protein total, |
| Induration | albumin, calcium, triglycerides, uric acid, |
| Adenopathy | lactate dehydrogenase (LDH), alkaline |
| Splenomegaly | phosphatase, creatine phosphokinase (CPK), |
| Adenopathy | amylase, creatinine, γ-glutamyl-trans- |
| Splenomegaly | peptidase (GGT). |

TABLE 16
Local effects, vital signs and alteration of hematological,
immunological and biochemical parameters upon each injection
of Tat protein formulated with Alum or H1D microparticles
in cynomolgus monkeys (Macaca fascicularis)

| | | | | | Modifications of |
|---|---|---|---|---|---|
| | | | | Modifi- | hematological, |
| | | | | cations | immunological |
| | | | Local | of vital | and biochemical |
| Mk | Immunogen | Route | effects | signs | parameters |
| L162F | Tat + Alum | s.c. | None | None | None |
| T197B | | | None | None | None |
| BA327C | | | None | None | None |
| M770F | Tat-H1D, | i.m., | None | None | None |
| O854G | Tat + Alum | s.c. | None | None | None |
| BD765B | | | None | None | None |
| AC032 | H1D, Alum | i.m., | None | None | None |
| AC739 | | s.c., | None | None | None |
| AC921 | | | None | None | None |

Measurement of Serum Antibodies Against the Tat Protein

For detection of anti-Tat antibodies, 96-well microplates (Nunc-Immuno Plate MaxiSorp Surface; Nunc) were coated with Tat protein (100 ng/200 μL per well, in 0.05 M carbonate buffer, pH 9.6) for 12 hrs at 4 ° C., and then washed 5 times with PBS without Ca²⁺ and Mg²⁺ containing 0.05% Tween 20 (PBS/Tween) on an automatic plate washer (Sorin Biomedica) to remove unbound Tat protein. Wells were then saturated with PBS containing 1% BSA and 0.05% Tween 20 (Sigma) (Blocking Buffer, BB) for 90 min at 37° C. After extensive washing, 100 μL of each serum sample diluted in BB (minimal serum dilution: 1:100) was added to the wells. To correct for any unspecific binding, each sample was always assessed in duplicate against both Tat and the buffer in which Tat had been resuspended.

In each experiment one known anti-Tat antibody positive sample and three known anti-Tat antibody negative samples were used as the positive and negative controls, respectively. After 90 min at 37 ° C. plates were extensively washed and wells were saturated with BB for 15 min at 37° C. Plates were the washed and 100 μL of an anti-monkey IgG horseradish peroxidase-conjugated secondary antibody (Sigma; diluted 1:1,000 in BB) were added to each well and incubated for additional 90 min at 37° C. After washing, antigen-bound antibodies were revealed by the addition of ABTS substrate solution (Roche Diagnostics) for 50 min at 37° C.

Absorbance was measured at 405 nm using a microplate reader (Sorin Biomedica). Optical densities (OD) of the samples were normalised for the background (buffer-coated well) of each sample. For each sample the OD difference between the wells coated with Tat and those coated with the buffer defined a Δ value. The assay was considered valid only when both the Δ values and the absolute values (before normalization) of the positive and negative controls were within ±10% variation with respect to values observed in previous 50 assays. Similarly, cut-off values were defined as 3 SD above the mean of both absolute OD and Δ values obtained with 50 samples from anti-Tat antibody negative monkey sera.

Lymphocyte Proliferation Assay

Ficoll-Hypaque (Pharmacia Bioteck AB, Uppsala, Sweden) gradient purified PBMCs were resuspended in complete RPMI medium complemented with 10% FCS, counted, seeded at 2×10⁵ cells per well in triplicate in 96-well microtiter plates and incubated for 5 days at 37 ° C. in 5% CO₂ in the absence or in the presence of either 5 μg/mL of Tat_{cys22} protein (HIV-1_{IIIB} mutant Tat lot: 4203, Advanced BioScience Laboratories, Inc, Rockville, Md.), or 2 μg/mL of a Tat peptides pool (15-mers overlapping by 10 residues) spanning the entire Tat sequence (aa 1-102). Phytohaemagglutinin (PHA, HA16, Murex Biotech, Dartford, UK) (2 μg/mL) was used as a positive control. At day 5, the cultures were pulsed for 16-18 hours with 1.0 μCi/well of [³H] thymidine (Amersham Bioscience, Uppsala, Sweden) and the incorporated radioactivity measured by a β-counter (Perkin-Elmer, Boston, Mass.). The stimulation index (S.I.) was calculated dividing the mean cpm values of stimulated samples by the mean cpm values of unstimulated samples. S.I.>3 were scored as positive.

IFN γ-ELISPOT Assay

The IFN γ-ELISpot assay was performed with reagents from Mabtech (Mabtech AB Gamla Värmdöv, Sweden) according to manufacturer's procedure. Briefly, PBMC isolated from monkeys were suspended in complete medium and seeded (2×10⁵/well, in duplicate) in a 96-well microtiter plate (MultiScreen-IP plate, Millipore Corporation, Bedford, Mass., USA) coated with a monoclonal antibody (mAb) against monkey IFN-γ (GZ4, mouse IgG1, Mabtech) in the presence of recombinant Tat_{cys22} (5 μg/mL) or of a pool of eighteen 15-mer Tat peptides (2 μg/mL of each peptide) spanning the whole protein. After overnight incubation at 37° C., cells were removed, and a biotinylated niAb against monkey IFN-γ (7-B6-1, mouse IgG1, Mabtech) were added to the wells. After 2 hours incubation at room temperature (RT), the plate was extensively washed and the Streptavidin-ALP (Alkaline Phosphatase, Mabtech) solution was added to the wells. After 60 min incubation at RT the plate was washed again and the chromogenic substrate BCIP/NBT (Sigma, Milan, IT) was added. After development (30-60 min at RT), spot forming cells (SFC) in each well were analyzed and counted by an ELISPOT reader (AID EliSpot Reader System, Autoimmun Diagnostika GmbH Strassberg, Germany or Automated ELISA-Spot Assay Video Analysis Systems®A.EL.VIS GmbH, Hannover, Germany) and expressed as SFC/10⁶ cells.

Results

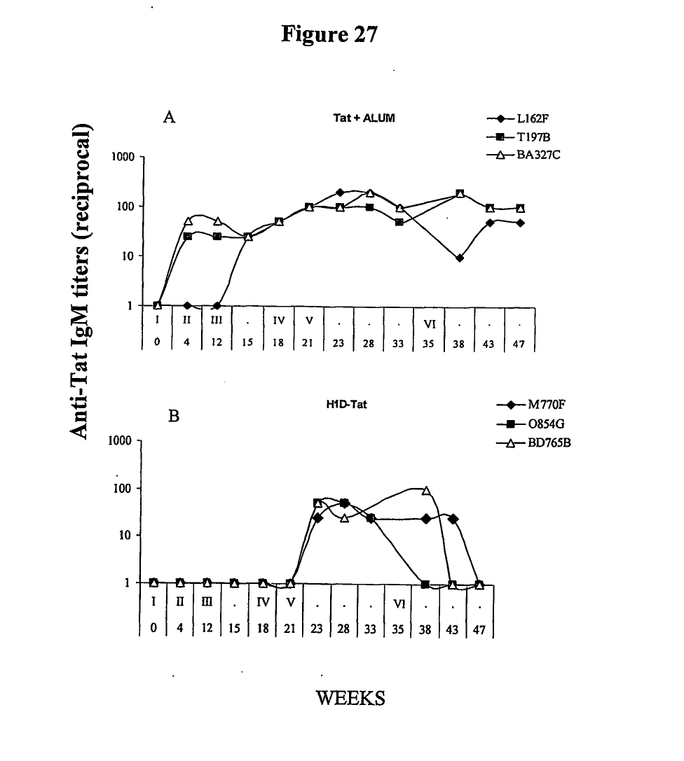

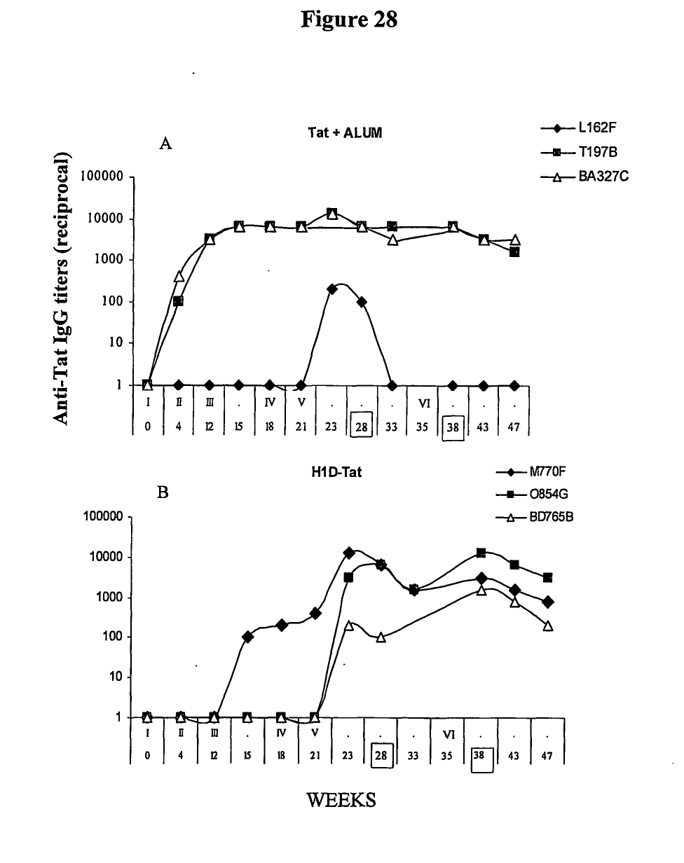

Results indicate that Tat protein adsorbed onto H1D microparticles (H1D-Tat) was effective at inducing both humoral and cellular immune responses although to a lesser extent than Tat+Alum immunization (FIGS. 27 to 29). In fact, as shown in FIGS. 27 and 28, panels D, E, and F, both IgM and IgG antibodies were measured in two of the three macaques immunized with H1D-Tat microparticles only after the first boost with Tat+Alum, a stimulus known to be optimal for the induction of Th2 responses and antibodies. The kinetic of appearance and peak antibody titers measured for both IgM and IgG in these two animals (FIGS. 27 and 28, panel E, and F) were similar to those observed in two out of three monkeys from group A (Tat+Alum) after the very first vaccine inoculation, indicating that H1D-Tat had not primed those 2 monkeys for antibody responses. However, in monkey M770F, immunized with H1D-Tat, IgG, but not IgM, were readily detected after the 3^{rd} H1D-Tat inoculation (FIGS. 27 and 28, panel D), indicating that this vaccine formulation is indeed capable to induce antibodies in monkeys, although only in a minority of the injected animals. The opposite occurred in group A macaques inoculated subcute with Tat and Alum (FIGS. 27 and 28, panels A, B, C), in which one of the three vaccinees mounted Ab responses only after the 4^{th} inoculum (FIGS. 27 and 28, panel A), whereas the remaining two did so after the 1^{st} vaccine administration (FIGS. 27 and 28, panels B and C), a finding in agreement with a certain heterogeneity of response to immunogens observed in outbred animals and in humans. Of note, while H1D-Tat was injected intramuscularly, Tat+Alum was administered subcutaneously. It remains to be determined whether the route of delivery had affected the induction of antibody responses. Overall, antibody responses were more robust in group A (Tat+Alum) macaques than in group B (H1D-Tat) animals (FIGS. 27 and 28).

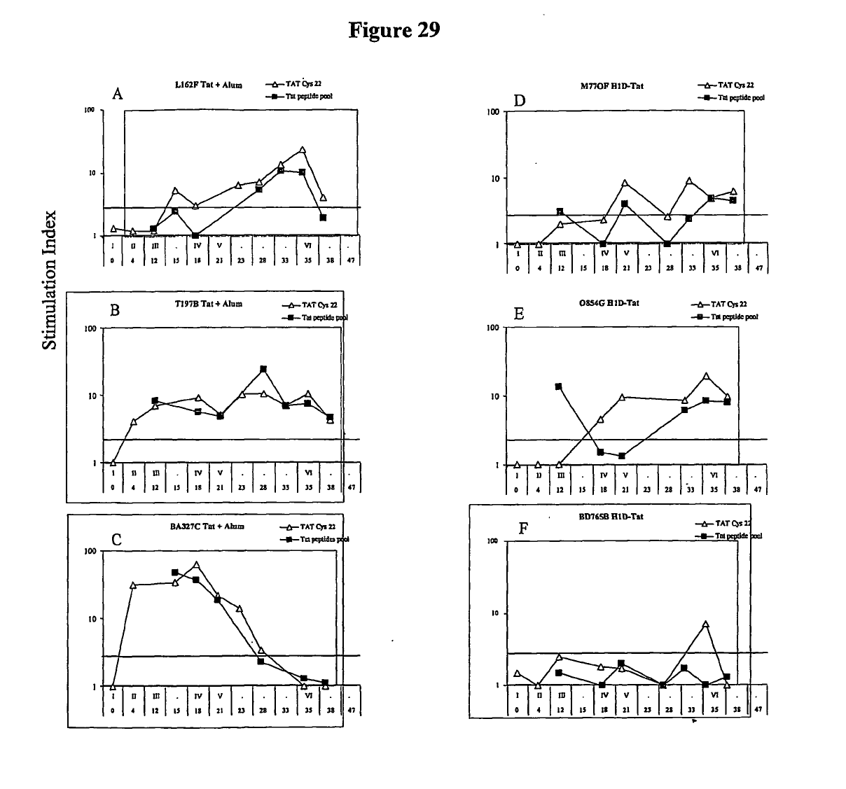

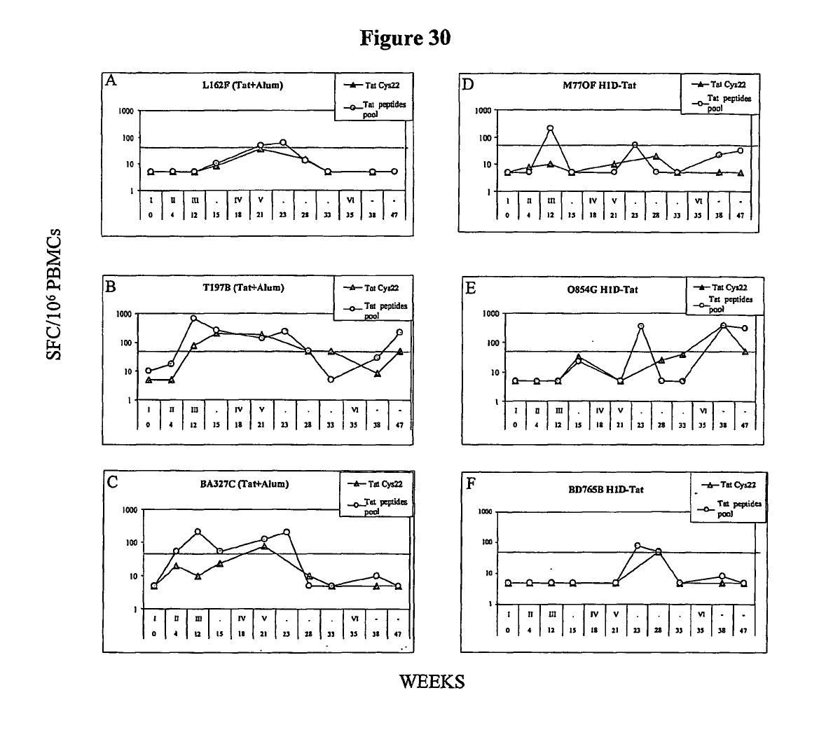

Lymphoproliferative responses are considered a good indicator of T helper responses for both B and T lymphocytes, a crucial event for the establishment of optimal and durable antibody and CTL responses. Therefore, T helper responses were measured utilizing as Tat antigen the Tat_{cys22} mutant or a pool of Tat peptides. This is because our previous data indicated that in monkeys the Tat_{wt} protein, but not the Tat_{cys22} mutant or a pool of Tat peptides, activates non-specifically T cell proliferation hampering measurement of specific responses. Proliferative responses to either Tat_{cys22} or a pool of Tat peptides were detected in monkeys vaccinated with H1D-Tat, although they were lower and less consistently detected as compared to those observed in macaques immunized with Tat+Alum (FIG. 29). A similar pattern of response was also observed when IFN-γ secreting cells in response to Tat_{cys22} mutant or a pool of Tat peptides were measured by Elispot assay (FIG. 30). As for the antibodies, the kinetic of appearance of cellular responses was somewhat delayed in the H1D-Tat group (FIGS. 41 and 42, panel B), as compared to the Tat+Alum group (FIGS. 29 and 30, panel A), especially when comparing proliferative responses (FIG. 29). Again, a certain variability in the response magnitude and durability was noted in both experimental groups (FIGS. 29 and 30, panel A and B). However, for each animal, in 5 out of 6 monkeys a good correlation was found among the different measurements of immune responses, strenghtening the significance of the findings (FIGS. 27 to 30).

In conclusion, these preliminary data indicate that intramuscular vaccination with H1D-Tat microparticles was safe and immunogenic in macaques. Additional studies evaluating the effect of antigen dose, route of administration, number of inocula, are needed to optimize H1D-Tat microparticles' immunogenicity.

1. A microparticle comprising:
(a) a core which comprises a water insoluble polymer or copolymer, and
(b) a shell which comprises a hydrophilic polymer or copolymer and functional groups which are ionic or ionisable;
said microparticle having a disease-associated antigen adsorbed at the external surface.

2. A microparticle according to claim 1, wherein the disease-associated antigen is a microbial antigen or a cancer-associated antigen.

3. A microparticle according to claim 1, wherein the water insoluble polymer is poly(styrene).

4. A microparticle according to claim 1, wherein the water insoluble polymer is poly(methylmethacrylate).

5. A microparticle according to claim 1, wherein the hydrophilic polymer is hemisuccinated polyvinylalcohol.

6. A microparticle according to claim 1, wherein the hydrophilic copolymer is Eudragit® L100-55 (a copolymer of methyacrylic acid and ethyl acrylate).

7. A microparticle according to claim 1, wherein the particle has a maximum size of from 0.1 to 10 μm.

8. A microparticle according to claim 1, wherein the antigen is a human immunodeficiency virus-1 (HIV-1) antigen.

9. A microparticle according to claim 8, wherein the antigen is HIV-1 Tat protein (SEQ ID NO: 2, 4, 6, 8, 10, 12, 14, 16, 18, 20, 22, 24, 26, 28, 30 or 32) or an immunogenic fragment thereof.

10. A method of production of a microparticle according to claim 1, said method comprising:
(a) polymerizing one or more water insoluble monomers in the presence of one or more hydrophilic polymer by dispersion polymerization to form microparticles; and
(b) adsorbing a disease-associated antigen at the external surface of said microparticles.

11. A pharmaceutical composition comprising a microparticle according to claim 1 and a pharmaceutically acceptable excipient.

12. A method of generating an immune response in an individual, said method comprising administering a microparticle according to claim 1 in a therapeutically effective amount.

13. A method according to claim 12, wherein the antigen is a human immunodeficiency virus-1 (HIV-1) antigen and the microparticle is administered to the individual to prevent or treat HIV infection or AIDS.

14-16. (canceled)
